(19) United States
(12) Patent Application Publication
(10) Pub. No.: US 2010/0209542 A1
(43) Pub. Date: Aug. 19, 2010

(54) Methods For Treating Withdrawal From Addictive Compounds

(75) Inventors: Edward W. Boyer, Needham, MA (US); Christopher Robert McCurdy, Oxford, MS (US); Jessica Erin Adkins, Pelzer, SC (US)

Correspondence Address: Peter G. Carroll;MEDLEN & CARROLL, LLP, Suite 350, 101 Howard Street, San Francisco, CA, 94105, US

(73) Assignees: University of Massachusetts Medical School; University of Mississippi

(21) Appl. No.: 12/623,064

(22) Filed: Nov. 20, 2009

Related U.S. Application Data
(60) Provisional application No. 61/116,912, filed on Nov. 21, 2008.

(51) Int. Cl. A61K 31/4375; A61K 36/185; A61P 25/30; A61P 25/36

(52) U.S. Cl. 424/725; 514/285

(57) ABSTRACT

Kratom (Mitragynia speciosa korth) is recognized increasingly as a remedy for opioid withdrawal by individuals who self-treat chronic pain and/or generalized substance abuse. For example, opioid withdrawal from an abrupt cessation of opiate abuse may be self-managed by using Kratom. Further, high-throughput molecular screening identified that a major component of Kratom (i.e., for example, mitragyinine) displayed high binding affinity at mu, delta and kappa opiate receptors. The present invention contemplates that Kratom extract may also be useful for the treatment of other addictive drugs besides opiate derivatives.

### STATEMENT OF GOVERNMENTAL SUPPORT

This invention was made with government support awarded by: i) the National Institutes of Health (grant number NIH 022677); ii) the National Institute For Drug Abuse (grant numbers DA022677 and DA014929); and iii) the National Center for Research Resources (grant number P20RR021929). The government has certain rights in the invention.

### FIELD OF THE INVENTION

This invention is related to the field of drug addiction and substance abuse. In particular, the invention utilizes compounds that facilitate reversal of drug addiction by avoiding the expression of a withdrawal syndrome. For example, natural compounds may be produced from the Kratom (Mitragyna speciosa Korth) leaf. Compounds isolated from Kratom leaf extracts may be capable of allowing a patient to cease the administration of addictive compounds without experiencing physically debilitating withdrawal symptoms.

### BACKGROUND

Drug addiction (i.e., for example, substance dependence) is usually exemplified by compulsively using a substance, despite its negative and sometimes dangerous effects. Drug abuse is commonly defined as using a drug excessively, or for purposes for which it was not medically intended. Behavioral patterns of addiction include compulsive drug-seeking, persistent abuse of substances despite the often dire consequences on social functioning and physical health, and the high probability of relapse even after prolonged drug-free periods.

The recent focus on the biological basis of drug addiction has provided evidence to support the hypothesis that behavioral manifestations for addiction are influenced by biological factors, and biological factors often produce behavioral changes that can further increase risk. The current understanding of the role of the dopaminergic, glutamatergic, epsilon-aminobutyric acidergic, and opioid receptor systems in the pathophysiology of addiction as well as the clinical implications of these systems for new and emerging treatments is improving. A variety of pharmacologic agents have been used in the treatment of substance abuse disorders and evaluated for safety, efficacy, and feasibility of use in different patient populations. Ivanov et al., “Neurorobiology and evidence-based biological treatments for substance abuse disorders” CNS Spectr. 11:864-877 (2006).

Nonetheless, clinical practice has not adequately dealt with the growing problem of treating subjects that have become addicted to compounds resulting from using either illicit drugs or legally prescribed pharmaceuticals. What is needed is a single agent capable of mitigating or otherwise treating addiction withdrawal where multiple agents with different CNS receptor binding affinities are presently required.

### SUMMARY

This invention is related to the field of drug addiction and substance abuse. In particular, the invention utilizes compounds that facilitate reversal of drug addiction by avoiding the expression of a withdrawal syndrome. For example, natural compounds may be produced from the Kratom leaf. Compounds isolated from Kratom leaf extracts may be capable of allowing a patient to cease the administration of addictive compounds without experiencing physically debilitating withdrawal symptoms.

In one embodiment, the present invention contemplates a method, a) providing, i) a subject experiencing at least one withdrawal symptom induced by at least one addictive compound; ii) a Kratom extract capable of reducing or preventing the at least one withdrawal symptom; and b) administering the Kratom extract to the subject under conditions such that the at least one withdrawal symptom is prevented and/or reduced. In one embodiment, the addictive compound comprises an opiate compound. In one embodiment, the addictive compound comprises an ethanol compound. In one embodiment, the addictive compound comprises a cocaine compound. In one embodiment, the addictive compound comprises a cannabinoid compound. In one embodiment, the opiate compound comprises a prescribed opiate compound. In one embodiment, the Kratom extract comprises mitragynine. In one embodiment, the Kratom extract comprises a mitragynine derivative. In one embodiment, the mitragynine derivative comprises mitragynine pseudoindoxyl or 7-hydroxymitragynine. In one embodiment, the withdrawal symptom comprises a craving induced by the addictive compound binding to a receptor selected from the group consisting of mu-, delta-, or kappa-opiate receptor.

In one embodiment, the present invention contemplates a method, a) providing, i) a subject repeatedly exposed to at least one compound, wherein the subject is at risk of experiencing at least one withdrawal symptom; ii) a Kratom extract capable of reducing or preventing the at least one withdrawal symptom; b) administering the Kratom extract as a substitute for the at least one compound under conditions such that the at least one withdrawal symptom is prevented or reduced. In one embodiment, the administering further comprises a regimen of decreasing said Kratom extract over a predetermined length of time. In one embodiment, the addictive compound comprises an opiate compound. In one embodiment, the addictive compound comprises an ethanol compound. In one embodiment, the addictive compound comprises a cocaine compound. In one embodiment, the addictive compound comprises a cannabinoid compound. In one embodiment, the opiate compound comprises a prescribed opiate compound. In one embodiment, the Kratom extract comprises mitragynine. In one embodiment, the Kratom extract comprises a mitragynine derivative. In one embodiment, the mitragynine derivative comprises mitragynine pseudoindoxyl or 7-hydroxymitragynine. In one embodiment, the withdrawal symptom comprises a craving induced by the addictive compound binding to a receptor selected from the group consisting of mu-, delta-, or kappa-opiate receptor.

In one embodiment, the present invention contemplates a method, a) providing, i) a subject experiencing at least one withdrawal symptom induced by at least one addictive compound; ii) a composition comprising mitragynine, wherein the composition is capable of reducing or preventing the at least one withdrawal symptom; and b) administering the composition to the subject under conditions such that the at least one withdrawal symptom is prevented and/or reduced. In one embodiment, the addictive compound comprises an opiate compound. In one embodiment, the addictive compound comprises an ethanol compound. In one embodiment, the addictive compound comprises a cocaine compound. In one embodiment, the addictive compound comprises a cannabinoid compound. In one embodiment, the opiate compound comprises a prescribed opiate compound. In one embodiment, the composition comprises a mitragynine derivative. In one embodiment, the mitragynine derivative comprises mitragynine pseudoindoxyl or 7-hydroxymitragynine. In one embodiment, the withdrawal symptom comprises a craving induced by the addictive compound binding to a receptor selected from the group consisting of mu-, delta-, or kappa-opiate receptor.

In one embodiment, the present invention contemplates a method, a) providing, i) a subject repeatedly exposed to at least one addictive compound, wherein the subject is at risk of experiencing at least one withdrawal symptom; ii) a composition comprising mitragynine, wherein the composition is capable of reducing or preventing the at least one withdrawal symptom; and b) administering the composition to the subject under conditions such that the at least one withdrawal symptom is prevented and/or reduced. In one embodiment, the administering further comprises a regimen of decreasing said composition comprising mitragynine over a predetermined length of time. In one embodiment, the addictive compound comprises an opiate compound. In one embodiment, the addictive compound comprises an ethanol compound. In one embodiment, the addictive compound comprises a cocaine compound. In one embodiment, the addictive compound comprises a cannabinoid compound. In one embodiment, the opiate compound comprises a prescribed opiate compound. In one embodiment, the composition comprises a mitragynine derivative. In one embodiment, the mitragynine derivative comprises mitragynine pseudoindoxyl or 7-hydroxymitragynine. In one embodiment, the withdrawal symptom comprises a craving induced by the addictive compound binding to a receptor selected from the group consisting of mu-, delta-, or kappa-opiate receptor.

In one embodiment, the present invention contemplates a method, a) providing, i) a subject experiencing at least one opioid withdrawal symptom induced by at least one addictive opioid compound; ii) a composition comprising mitragynine, wherein the composition is capable of reducing or preventing the at least one withdrawal symptom; and b) administering the composition to the subject under conditions such that the at least one withdrawal symptom is prevented and/or reduced. In one embodiment, the opiate compound comprises a prescribed opiate compound. In one embodiment, the composition comprises a mitragynine derivative. In one embodiment, the mitragynine derivative comprises mitragynine pseudoindoxyl or 7-hydroxymitragynine. In one embodiment, the withdrawal symptom comprises a craving induced by the addictive compound binding to a receptor selected from the group consisting of mu-, delta-, or kappa-opiate receptor.

In one embodiment, the present invention contemplates a method, a) providing, i) a subject repeatedly exposed to at least one addictive opioid compound, wherein the subject is at risk of experiencing at least one opioid withdrawal symptom; ii) a composition comprising mitragynine, wherein the composition is capable of reducing or preventing the at least one withdrawal symptom; and b) administering the composition to the subject under conditions such that the at least one withdrawal symptom is prevented and/or reduced. In one embodiment, the administering further comprises a regimen of decreasing said composition comprising mitragynine over a predetermined length of time. In one embodiment, the opiate compound comprises a prescribed opiate compound. In one embodiment, the composition comprises a mitragynine derivative. In one embodiment, the mitragynine derivative comprises mitragynine pseudoindoxyl or 7-hydroxymitragynine. In one embodiment, the withdrawal symptom comprises a craving induced by the addictive compound binding to a receptor selected from the group consisting of mu-, delta-, or kappa-opiate receptor.

### DEFINITIONS

The term “abuse” as used herein, refers to any intentional use of opioids outside of a physician's prescription for a bona fide medical condition, excluding accidental misuse.

The term “addiction” as used herein, refers to any neurobehavioral syndrome characterized by compulsive use, impaired control, tolerance, withdrawal, and continued use despite physical and psychological problems caused or exacerbated by use.

The term “dependence” as used herein, refers to any physiological state of adaptation to opioid analgesics, the absence of which produces signs and symptoms of withdrawal.

The term “tolerance” as used herein, refers to the need to use more opioid than previously to achieve the same effect. For example, the tolerance may comprise “functional” tolerance where the target tissue becomes relatively resistant to the drug (i.e., for example, by drug receptor up-regulation and/or decrease in receptor drug affinity). Alternatively, the tolerance may comprise “metabolic” tolerance where the drug is simply degrated faster (i.e, for example, by an increase in degradative enzymes).

The term “withdrawal” as used herein, refers to any predictable constellation of signs and symptoms resulting from abrupt removal of, or a rapid decrease in the regular dosage of an opioid.

The term “chronic pain” as used herein, refers to any persistent pain of greater than six (6) months' duration.

The term “drug” or “compound” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides or sugars.

The term “administered” or “administering” a drug or compound, as used herein, refers to any method of providing a drug or compound to a patient such that the drug or compound has its intended effect on the patient. For example, one method of administering is by an indirect mechanism using a medical device such as, but not limited to a catheter, applicator gun, syringe etc. A second exemplary method of administering is by a direct mechanism such as, local tissue administration (i.e., for example, extravascular placement), oral ingestion, transdermal patch, topical, inhalation, suppository etc.

The term “substitute for” as used herein, refers to switching the administration of a first compound or drug to a subject for a second compound or drug to the subject. For example, a Kratom extract may be substituted for an addictive compound such that a subject will be administered the Kratom extract instead of the addictive compound.

The term “repeatedly exposed” as used herein, refers to the administration of an addictive drug or compound to a subject on a regular schedule for a prolonged period of time. Such a schedule may comprise administration of a dose ranging between approximately once-to-twelve times per day for a time period lasting at least two successive days and may range over weeks, months, years, and even decades.

The term “at risk for” as used herein, refers to a medical condition or set of medical conditions exhibited by a patient which may predispose the patient to a particular disease or affliction. For example, these conditions may result from influences that include, but are not limited to, behavioral, emotional, chemical, biochemical, or environmental influences.

The terms “reduce,” “inhibit,” “diminish,” “suppress,” “decrease,” “prevent” and grammatical equivalents (including “lower,” “smaller,” etc.) when in reference to the expression of any symptom (e.g., a withdrawal symptom) in an untreated addicted subject relative to a treated addicted subject, mean that the quantity and/or magnitude of the symptoms in the treated addicted subject is lower than in the untreated addicted subject by any amount that is recognized as clinically relevant by any medically trained personnel. In one embodiment, the quantity and/or magnitude of the symptoms in the treated addicted subject is at least 10% lower than, at least 25% lower than, at least 50% lower than, at least 75% lower than, and/or at least 90% lower than the quantity and/or magnitude of the symptoms in the untreated addicted subject.

The term “patient” or “subject”, as used herein, is a human or animal and need not be hospitalized. For example, out-patients, persons in nursing homes are “patients.” A patient may comprise any age of a human or non-human animal and therefore includes both adult and juveniles (i.e., children). It is not intended that the term “patient” connote a need for medical treatment, therefore, a patient may voluntarily or involuntarily be part of experimentation whether clinical or in support of basic science studies.

The term “affinity” as used herein, refers to any attractive force between substances or particles that causes them to enter into and remain in chemical combination. For example, an inhibitor compound that has a high affinity for a receptor will provide greater efficacy in preventing the receptor from interacting with its natural ligands, than an inhibitor with a low affinity.

The term “effective amount” as used herein, refers to a particular amount of a pharmaceutical composition comprising a therapeutic agent (i.e., for example, an opiate receptor agonist or antagonist) that achieves a clinically beneficial result.

The term “derived from” as used herein, refers to the source of a compound or drug. In one respect, a compound or drug may be derived from an organism or particular species. In another respect, a compound or drug may be derived from a larger macromolecular complex.

The term “pharmaceutically” or “pharmacologically acceptable”, as used herein, refer to molecular entities and compositions that do not produce adverse, allergic, or other untoward reactions when administered to an animal or a human.

The term, “purified” or “isolated”, as used herein, may refer to a composition or compound (i.e., for example, a chemical compound) that has been subjected to treatment (i.e., for example, chromatography) to remove various other components, and which composition substantially retains its expressed biological activity. Where the term “substantially purified” is used, this designation will refer to a composition in which the compound forms the major component of the composition, such as constituting about 50%, about 60%, about 70%, about 80%, about 90%, about 95% or more of the composition (i.e., for example, weight/weight and/or weight/volume). A purified composition is not intended to mean that some trace impurities may remain.

As used herein, the term “substantially purified” refers to molecules, either compounds or drugs, that are removed from their natural environment, isolated or separated, and are at least 60% free, preferably 75% free, and more preferably 90% free from other components with which they are naturally associated. An “isolated compound or drug” is therefore a substantially purified compound or drug.

The term “small organic molecule” as used herein, refers to any molecule of a size comparable to those organic molecules generally used in pharmaceuticals. The term excludes biological macromolecules (e.g., proteins, nucleic acids, etc.). Preferred small organic molecules range in size from approximately 10 Da up to about 5000 Da, more preferably up to 2000 Da, and most preferably up to about 1000 Da.

The term “derivative” as used herein, refers to any chemical modification of a compound or drug. Illustrative of such modifications would be replacement of hydrogen by an alkyl, acyl, or amino group. For example, an opiate derivative would result in a different compound which retains essential biological characteristics such as binding affinity to an opiate receptor.

The term “biologically active” refers to any molecule or compound having structural, regulatory or biochemical functions.

The term “binding” as used herein, refers to any interaction between at least two different compounds. Binding may be reversible or irreversible. Such binding may be, but is not limited to, non-covalent binding, covalent bonding, ionic bonding, Van de Waal forces or friction, and the like.

The term “opiate compound” as used herein, refers to any compound having a morphine-based ring structure such that the structure-activity relationships of the compound results in physiological binding affinity to an opiate receptor (i.e., for example, K_{d}=10⁻³ to 10⁻¹² M.). Such opiate compound may include, but are not limited to, heroin, opium, codeine, meperidine (i.e., for example, Demerol®), hydromorphone (i.e., for example, Dilaudid®), oxycontin, hydrocodone, oxycodone, fentanyl, morphine, methadone, and tramadol.

The term “ethanol compound” as used herein, refers to any compound or composition comprising ethanol (i.e, for example, CH₃CH₂OH). Ethanol is usually found in commercially available beverages including, but not limited to, beer, wine, and distilled liquors. An ethanol compound may comprise a variable concentration of ethanol ranging between 3%-100%, preferably ranging between 6%-50%, but more preferable ranging between 12%-25%.

The term “cocaine compound” as used herein, refers to any compound or composition comprising cocaine a derivative thereof, or an extract of a coca leaf. For example, cocaine may be processed into various compositions that comprise an altered chemical structure and/or crystalline structure. Such alterations may change pharmacokinetic parameters and/or usable routes of administration that may facilitate the development of cocaine addiction.

The term “nociceptive neurons” or “nociceptors” as used herein, refers to any neurons which respond to stimuli that are damaging or potentially damaging to the skin (e.g., intense pressure, high heat, and burning chemicals), and which thereby mediate pain.

The term “analgesia” or “relief from pain” as used herein, results from activation by opioid agonists of inhibitory opioid receptors on neurons in the nociceptive (pain) pathways of the peripheral and central nervous systems.

The term “psychological dependence” as used herein, refers to any psychological condition which manifests as an overpowering compulsion to continue taking an addictive drug (i.e., for example, an opioid).

The term “physical dependence” as used herein, refers to a state of physiologic adaptation to a drug, which may increase in intensity with increased dosage and duration of use of an addictive drug, and which may manifest in a withdrawal (abstinence) syndrome when the drug is discontinued or its effect is counteracted.

The term “tolerance” as used herein, refers to circumstances where the dosage of an addictive drug must be increased in order to obtain the initial effect.

### BRIEF DESCRIPTION OF THE FIGURES

FIG. 1 presents Panel A showing the basic indole ring structure of components of an alkaloid Kratom extract and Panel B presenting the structure of mitragynine and identifies several structure-activity relationships.

FIG. 2 presents exemplary data showing plasma concentration-time curves for a single 20 mg/kg mitragyinine dose administered by oral gavage in Wistar rats.

FIG. 3 presents exemplary data demonstrating assay validation for the detection of mitragynine.

FIG. 4 presents exemplary structures of common long-chain bases from plants. All naturally occurring dihydroxy sphingoid bases have D-erythro and all trihydroxy sphingoid bases have D-ribo configuration. Additional double bonds and hydroxy groups characterizing the sphingoid bases derived from sphinganine are marked by grey background. While C₁₈-sphingoid bases are depicted, long-chain bases of different chain lengths occur in minor amounts. For example, in plant cerebrosides, the 8-(E)- and 8-(Z)-isomers of sphinga-4,8-dienine and 4-hydroxy-sphinga-8-enines are dominant, whereas sphingosine and 4-hydroxysphinganine, are predominant long-chain bases in animals and S. cerevisiae.

FIG. 5 presents exemplary structures of ceramide glycosides from plants. The ceramide backbones of plant sphingolipids show a large variability. These long-chain bases (LCBs) are presented in four embodiments (on grey background); i) hydroxylation; ii) (E)-desaturation at C-4, iii) (E)-desaturation at C-8; and iv) (Z)-desaturation at C-8. These LCB are linked to more than 10 different fatty acyl groups, which in turn vary in α-hydroxylation, chain length and Δ9-unsaturation. Cerebrosides are formed by glycosylation of the C1-hydroxy group of ceramide (R₁) yielding β-D-glucosyl and β-D-mannosyl ceramide (R₂₋₃). The glucosyl derivative may by elongated by sequential addition of up to three β-1,4-mannosyl residues resulting in oligosaccharides terminally capped by β-1,4-glucosyl residue (R₄₋₅). Glycosyl inositol phosphorylceramides (GIPC, phytoglycolipids) isolated from tobacco leaves (R₆₋₇) and corn kernels (R₈) carry a 1-phosphomyo-inositol residue linked via C2 and/or C6 to C1 of an a-glucuronosyl residue. Additional glycosyl derivatives with further galactosyl, mannosyl and fucosyl residues have been isolated from R₇₋₈.

FIG. 6 presents one embodiment of a pathway for sphingolipid biosynthesis in plants. Metabolites are shown in bold lettering, enzymes and their genes are included on grey background. Genes occurring in S. cerevisiae are given in the preferred designations listed in the Saccharomyces Genome Database. Functionally identified plant genes are marked by black framing of the enzymes. The presence of putative A. thaliana genes identified by comparison of their deduced amino acid sequences is indicated by a black dot next to the enzyme. The placement of some steps of sphingolipid synthesis, in particular of Δ4- and Δ8-LCB desaturation, the substrate specificities of the involved enzymes (i.e. free ceramide, cerebroside and GIPC) and the channelling of molecular ceramide species into complex sphingolipids are hypothetical (infra).

FIG. 7 illustrates a possible phylogram showing similarities between sphingolipid desaturases/hydroxylases and selected Δ4/Δ5/Δ6-fatty acyl desaturases. The regio-selectivities of lipid desaturases are indicated by Δ-numbers, their stereoselectivity by (E), (Z) or (E/Z) and the presence of a N-terminal cytochrome b5 fusion domain by dotted lines. All sphingolipid modifying enzyme groups are marked by grey background. Note the close similarity of the Δ6-fatty acyl desaturases from B. officinalis and Mucor rouxii to the Δ8-LCB desaturases. Functionally identified enzymes are highlighted by bold lettering. Superscripts refer to the following Accession numbers: ¹AF02104, ²Z81038, ³Z97209, ⁴CAD21081, ⁵Z49260, ⁶AAA16608, ⁷C. albicans ORF 6.4041 on contig 6-2307, ⁸CAA21900, ⁹AC013289, ¹⁰AC012188, ¹¹X87094, ¹²L11421, ¹³unpublished, ¹⁴AF489589, ¹⁵AF489588, ¹⁶AJ250734, ¹⁷AJ250735, ¹⁸AJ222980, ¹⁹AF296076, ²⁰unpublished, ²¹C. albicans genomic sequence on contig 6-1607, bases 2181430, ²²BAB93118, ²³BAB93117, ²⁴unpublished, ²⁵AF031194, ²⁶U79010, ²⁷AF406816, ²⁸AF133728, ²⁹×87143, ³⁰AJ224161, ³¹AJ224160, ³²AC005397, ³³BAB58879, ³⁴Neurospora crassa genomic sequence on contig 9a58, bases 17916-16535, ³⁵T40333, ³⁶C. albicans genomic sequence on contig 6-2340, bases 7499-8611, ³⁷unpublished, ³⁸AAD17340, ³⁹AF466378, ⁴⁰AF466379, ⁴¹AF466377, ⁴²AF466375, ⁴³AF466376, ⁴⁴NP_{—}445775, ⁴⁵NP_{—}501256, ⁴⁶NP_{—}493549.

### DETAILED DESCRIPTION OF THE INVENTION

This invention is related to the field of addiction and substance abuse. In particular, the invention utilizes compounds that reverse addiction while avoiding the expression of a withdrawal syndrome. For example, such natural compounds may be produced in the Kratom leaf. Compounds isolated from Kratom leaf extracts may be capable of allowing a patient to cease the administration of addictive compounds without experiencing physically debilitating withdrawal symptoms.

Drugs of abuse have in common the fact that they serve as biological rewards. As a result, any abused drug may be capable of activating specific endogenous brain neuronal pathways. Identification of such common neuronal pathways may find a common basis for the abuse liability of seemingly different addictive drugs. For example, two classes of abused drugs, psychomotor stimulants and opiates, may share a single brain reward mechanism. Some have suggested this common pathway to comprise dopamine-containing cells of the ventral tegmental area and their fiber projections to the cells of the nucleus accumbens. Morphine activates these cells possibly mediated via receptors imbedded in dopaminergic cell body membranes, or it may act on afferent terminals that synapse on the dopaminergic cell bodies or dendrites. Stimulants (i.e, for example, cocaine or amphetamine) may act at the terminals of the dopaminergic fibers to nucleus accumbens and perhaps other structures. The shared activation of the dopaminergic input to nucleus accumbens accounts for the behaviorally activating and the rewarding effects of both stimulants and opiates (the opiate stimulant action is not widely known because it is usually masked by depressant actions of opiates in other, antagonistic, brain circuits). The activation of dopaminergic systems may also account for amphetamine euphoria, cocaine euphoria, and opiate euphoria. Wise et al., “Brain mechanisms of drug reward and euphoria” Psychiatr Med 3:445-460 (1985). Recent studies, however, have identified the possible involvement of the specific opioid receptors in cocaine addiction and alcoholism. Kreek, M. J., “Opioid receptors: some perspectives from early studies of their role in normal physiology, stress responsivity, and in specific addictive diseases” Neurochem Res. 21:1469-88 (1996); and Kreek, M. J., “Opiates, opioids and addiction” Mol Psychiatry 1:232-54 (1996).

Drug abuse can lead to drug dependence and/or addiction. People who use drugs for pain relief may become dependent, although this is rare in those who don't have a history of addiction. A physical dependence on a substance (i.e., needing the drug to function) is not always part of the definition of addiction. Some drugs (for example, some blood pressure medications) don't cause addiction but do cause physical dependence. Other drugs may cause addiction without physical dependence. For example, cocaine withdrawal may not express symptoms like vomiting and chills (i.e., for example, physical symptoms) but usually involves depression (i.e., for example, behavioral symptoms).

The exact cause of drug abuse and dependence is not known. However, a person's genes, the action of a drug, peer pressure, emotional distress, anxiety, depression, and environmental stress all can be factors. Peer pressure can lead to drug use or abuse, but at least half of those who become addicted have depression, attention deficit disorder, post-traumatic stress disorder, or another psychological problem.

Symptoms of drug abuse may include, but are not limited to, a unexplained change in friends, withdrawn behavior, long unexplained periods away from home, lying, stealing, unusual interaction with legal authorities, compromised family relations, acting drunk or high (intoxicated), confusion, impossible to understand, unconsciousness, distinct changes in behavior and normal attitude.

Commonly abused substances include, but are not limited to, opiates and/or narcotics including, but not limited to, heroin, opium, codeine, meperidine (Demerol®), hydromorphone (Dilaudid®), and Oxycontin®; central nervous system (CNS) stimulants including, but not limited to, amphetamines, cocaine, dextroamphetamine, methamphetamine, methylphenidate (Ritalin®), caffeine or nicotine; CNS depressants including, but not limited to, barbiturates (i.e., for example, amobarbital, pentobarbital, or secobarbital), benzodiazepines (i.e., for example, Valium®, Ativan®, Xanax®), chloral hydrate, or paraldehyde; ethanol; hallucinogens including, but not limited to, lysergic acid diethylaminde (LSD), mescaline, psilocybin (i.e., for example, “magic” mushrooms), and phencyclidine (PCP or “Angel Dust”); or tetrahydrocannabinol (THC) as the active ingredient found in marijuana (Cannabis sativa) and hashish.

Drug withdrawal symptoms can occur when a person stops or reduces their use of a substance. Withdrawal symptoms vary, depending on the abused substance. When withdrawal symptoms begin depends on the length of time the drug normally stays within the body. Drug intoxication, overdose, and withdrawal can sometimes be life-threatening.

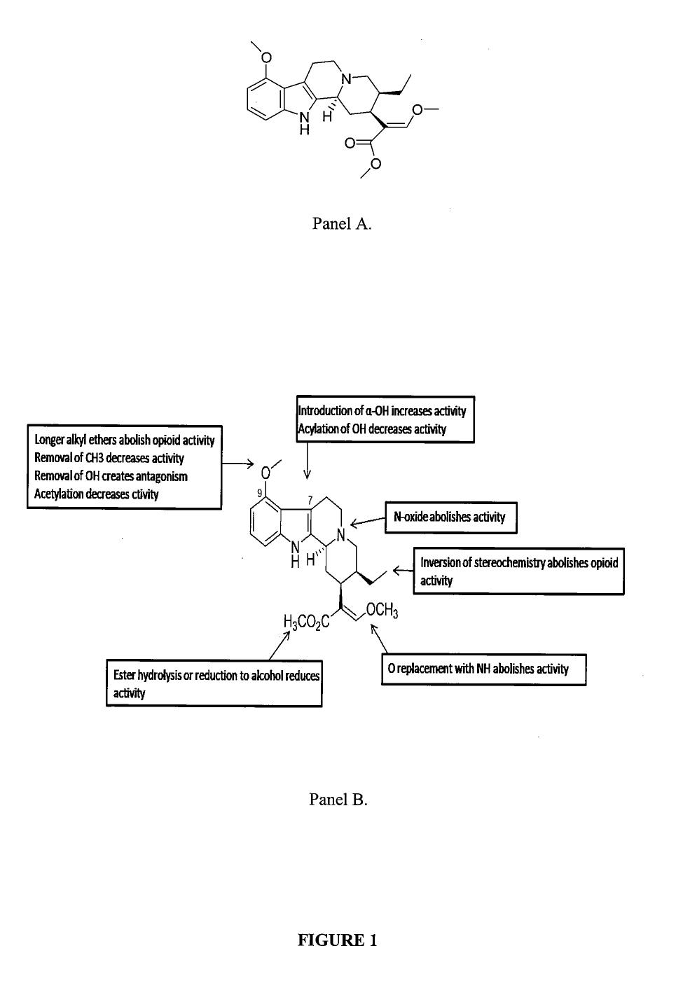

In one embodiment, the present invention contemplates a method for the treatment of withdrawal from an addictive drug. In one embodiment, the present invention contemplates a composition comprising a Kratom leaf extract. Although it is not necessary to understand the mechanism of an invention, it is believed that Kratom is a medicinal leaf harvested from Mitragyna speciosa, a tree native to Southeast Asia. In one embodiment, the Kratom leaf extract comprises mitragynine. See, FIG. 1. Although it is not necessary to understand the mechanism of an invention, it is believed that mitragynine might be the most abundant alkaloid in Kratom leaves. The compound is believed to bind to, inter alia, μ- and κ-opiate receptors. Although it is not necessary to understand the mechanism of an invention, it is believed that a mitragynine-receptor interaction is presumably responsible for the treatment of addictive drug withdrawal.

### I. Opioid Addiction and Withdrawal

In one embodiment, the present invention contemplates a method of treating opioid withdrawal syndrome using a Kratom extract or compounds derived from a Kratom extract. Opioid addiction can result from either illicit recreational use (where the subject obtains the drug “on the street”) or from a legal drug prescription received from a medical doctor. Such legal prescriptions are routinely provided for conditions including, but not limited to, chronic pain. Pain therapy routinely involves the prescription of opioid compounds, wherein natural compounds may provide a reversal of the addiction phenomenon while sparing the patient the clinical manifestations of a classical withdrawal syndrome.

Symptoms of opiate and/or narcotic use include, but are not limited to, needle marks on the skin in some cases (called “tracks”), scars from skin abscesses, rapid heart rate, small pupils (pinpoint), relaxed and/or euphoric state (“nodding”), coma, respiratory depression leading to coma, and death in high doses.

Symptoms of opiate and/or narcotic withdrawal include, but are not limited to, anxiety and difficulty sleeping, sweating, goose bumps (piloerection), runny nose (rhinorrhea), stomach cramps and/or diarrhea, enlarged (dilated) pupils, nausea and/or vomiting, excessive sweating, increase in blood pressure, pulse, and/or temperature.

The World Health Organization (WHO) analgesic ladder principle continues to serve as an educational tool in the efforts by WHO in collaboration with the World Federation of Societies of Anaesthesiologists (WFSA) and The International Association for the Study of Pain (IASP) to increase knowledge of pharmacological pain therapy and increase availability of essential opioid analgesics world-wide. Opioids differ in pharmacodynamics and pharmacokinetics, and patients have different pharmacogenetics and pain mechanisms. Sequential trials of the increasing numbers of available opioid drugs are therefore appropriate when oral morphine fails.

Controversies continue concerning diagnosis and handling of: i) opioid-insensitive pain in both chronic cancer and chronic non-cancer pain; ii) opioid-induced neurotoxicities; iii) risks of tolerance, addiction, pseudo-addiction; iv) methods for improving effectiveness and decreasing adverse effects of long-term opioid therapy; and v) treating breakthrough pain with immediate release oral and transmucosal opioids. Consensus guidelines have recently been developed in the Nordic countries concerning the ethical practice of palliative sedation when opioids and other pain-relieving therapies fail in patients soon to die. Guidelines for long-term treatment with strong opioids of chronic non-cancer-related pain are also being developed in the Nordic countries, where very diverging traditions for the usage of such therapy still exist.

Opioid analgesics remain highly effective modalities for the treatment of chronic pain, but their long-term administration is associated with the development of opioid misuse, abuse, dependence and addiction, the incidence of which is increasing. Fishbain et al., “Drug abuse, dependence, and addiction in chronic pain patients” Clin J Pain 8:77-85 (1992).

Developing pharmacological treatments for opioid dependence and withdrawal that possess potential added benefits over the existing interventions is of great importance. Opioid replacement therapies commonly involve methadone or Suboxone® (a buprenorphine/naloxone co-formulation). Both of these therapies suffer from significant limitations. Dramatic increases in accidental deaths from methadone correlate with increased rates of its prescription.⁴Suboxone®, a more recent therapeutic development, suffers from poor penetration into communities of opioid addicts because of severe regulatory restriction on the number of prescriptions that physicians may write. Furthermore, Suboxone® is contraindicated in individuals who abuse Oxycontin® or fentanyl patches, two of the most commonly abused prescription opioid analgesic formulations. Lastly, Suboxone®, a treatment for addiction, has poor acceptance among individuals who self-treat chronic pain with opioid analgesics purchased from Internet pharmacies, a population who does not view themselves as addicts.¹ An urgent need therefore exists to develop effective and safe pharmacologic interventions for prescription opioid analgesic addiction.

Abuse of, and addiction to, opioid agents is not a new phenomenon.¹⁰ What is unprecedented, however, is the scale, range and growth of the abuse of opioid analgesic agents. In addition, a marked increase in the therapeutic use of opioid medications has been observed in the United States along with an even greater increase in problems associated with these agents' use.¹¹ The surging use of opioids and associated problems is particularly concerning because it represents an expanded pathway to opioid addiction.^{12, 13}

Between 1999 and 2002, the number of opioid analgesic poisonings on death certificates increased 91.2%, while heroin and cocaine poisonings increased 12.4% and 22.8%, respectively. The increase in deaths generally matched the increase in sales for each type of opioid.⁴ In 2002 ˜4.7% of American household residents over age 12 had abused an opioid medication; 13.7% of these individuals met DSM-IV criterion for a diagnosis of opioid abuse disorder.^{14, 15} Hydrocodone has been reported to rank as the second-most abused substance among college students, a population noted for exploratory substance use. Risk factors associated with tolerance, dependence, and abuse of opioid analgesics are poorly understood.^{13, 16-18} Prescription opioid analgesics that are commonly abused in the United States include, but are not limited to, hydrocodone, oxycodone, hydromorphone, codeine, fentanyl, morphine, methadone, and tramadol.

Further, methadone deaths increased nearly 500% between 1999 and 2005.¹⁹ Dramatic increases in deaths from methadone correlate with increased rates of prescription.⁴ Most deaths occur soon after initiation of methadone treatment in chronic pain patients, and not from methadone-based treatment of heroin addiction.⁴ Clusters of methadone deaths in North Carolina and New Mexico overlay an increasing baseline of methadone deaths nationally.^{20, 21} Suboxone® produces life-threatening toxicity as well. Ingestion of Suboxone® as a result of normal childhood exploratory behavior has led to coma, apnea, and severe adverse effects including cortical blindness. Importantly, children need not ingest a Suboxone® tablet; merely putting a tablet in the mouth is sufficient to produce life-threatening toxicity.²²

One reason for the higher death rate among opioid dependent individuals with chronic pain is because they do not view themselves as addicts. In their mind, addicts cannot function because they use drugs. The opposite, however, is true. An addict must use the drug in order to function (i.e., feel normal). It is in the absence of drug use where an addict begins to have trouble functioning and ultimately encounters symptoms of a withdrawal syndrome.

Opioid-dependent chronic pain suffers, particularly those who self-prescribe hydrocodone and/or oxycodone, also believe that they function because they use drugs. To the extent this is true, methadone is a highly unacceptable medication because of its association with heroin addict and treatment. Suboxone® has met with greater acceptance by this population, but the limitation on the number of prescriptions that a clinician may write has dampened the enthusiasm of prescription opioid abusers for this therapy.

### II. Cocaine Addiction and Withdrawal

In one embodiment, the present invention contemplates a method of treating cocaine withdrawal syndrome using a Kratom extract or compounds derived from a Kratom extract. Cocaine addiction results primarily from illicit recreational use where the subject obtains the drug “on the street”, wherein natural compounds may provide a reversal of the addiction phenomenon while sparing the patient the clinical manifestations of a classical withdrawal syndrome.

Symptoms of cocaine use includes, but are not limited to, exaggerated feeling of well-being (euphoria), dilated pupils, fast heart rate, or restlessness and/or hyperactivity.

Symptoms of cocaine withdrawal includes, but are not limited to, fatigue and/or malaise, depression, and very clear and/or unpleasant dreams.

Many studies have shown interactions between mu-opiates and the mesolimbic dopamine (DA) system. For example, mu-opiate receptor antagonists have been reported to modulate the rate of cocaine self-administration. Characterization and localization the effect of opiate receptor blockade on the reinforcing effects of cocaine was studied using the mu-opiate irreversible antagonist beta-funaltrexamine (betaFNA). Microinjection of betaFNA into the ventral tegmental area (VTA) or the nucleus accumbens (NAcc) had no effect on cocaine self-administration under a fixed ratio (FR) schedule of reinforcement. However, blockade of opiate receptors in both brain regions did attenuate responding for cocaine maintained by a progressive ratio (PR) schedule. Administration of betaFNA in the dorsal striatum had no effect under either schedule condition. These data suggest that endogenous opiate systems within the mesolimbic DA system modulate the reinforcing effects of cocaine; however, this modulation seemed to be schedule dependent. Ward et al., “Beta-funaltrexamine affects cocaine self-administration in rats responding on a progressive ratio schedule of reinforcement” Pharmacol Biochem Behav. 75:301-307 (2003).

Numerous reports support evidence that dopaminergic mesolimbic pathways interact with opioid systems to influence the reinforcing properties of cocaine. For example, withdrawal from chronic administration of cocaine in rats causes an upregulation of mesocorticolimbic mu-opioid receptors during early stages. Prolonged cocaine abstinence was addressed by treating rats with cocaine or saline (control) intermittently (1 mg/kg, i.v., every 12 min for 2 h daily) for 10 days followed by a 10- or 20-day withdrawal period following which a quantitative in vitro autoradiographic analysis of 14 brain regions with (125)I-DAMGO was performed. A separate group of animals received two consecutive cycles of the 10-day cocaine/10-day withdrawal regimen. Only the group that participated in the two consecutive cycles showed a significant effect of cocaine treatment by a downregulation of mu-opiate receptors in; i) the limbic cortical layer 3 (17% lower than saline-treated controls, P=0.03); ii) the core of the nucleus accumbens (16% decrease, P=0.05); and iii) the nucleus of the diagonal band (18% decrease, P=0.05). Sharpe et al., “Autoradiographic evidence that prolonged withdrawal from intermittent cocaine reduces mu-opioid receptor expression in limbic regions of the rat brain” Synapse 37:292-297 (2000).

Opiate-related molecular changes were found in the neostriatum of human subjects who died with a history of cocaine abuse and had detectable cocaine and/or cocaine metabolites at the time of death. Marked reductions in the levels of enkephalin mRNA and mu opiate receptor binding were found in the caudate and putamen, concomitant with elevations in levels of dynorphin mRNA and kappa opiate receptor binding in the putamen and caudate, respectively. Additionally, an imbalance in the activity of the two major striatal output pathways in cocaine users is implicated because peptide mRNA levels were reduced in enkephalinergic striatopallidal neurons and increased in dynorphinergic striatonigral neurons. Another imbalance, that of reductions of transmitter mRNA and receptor expression associated with euphoria (enkephalin and mu opiate receptors), together with elevations in mRNAs of transmitter systems associated with dysphoria (dynorphin and kappa opiate receptors), suggests a model of dysphoria and craving in the human cocaine addict brain. Hurd et al., “Molecular alterations in the neostriatum of human cocaine addicts” Synapse 13:357-369 (1993).

In vitro receptor autoradiography was used to determine the effect of chronic, continuous cocaine exposure of 2 weeks duration on [³H]naloxone binding in various regions of rat brain. Although cocaine action in the central nervous system is usually associated with altered dopamine function, opiate receptor density (measured via [³H]naloxone) was altered by chronic cocaine exposure in critical brain reward regions, including the nucleus accumbens, ventral pallidum, and lateral hypothalamus. Endogenous opioid activity at opiate receptors in these critical regions may be associated with the reinforcement induced by both cocaine and opiates. Hammer R. P., “Cocaine alters opiate receptor binding in critical brain reward regions” Synaps. 3:55-60 (1989).

### III. Ethanol Addiction and Withdrawal

In one embodiment, the present invention contemplates a method of treating ethanol withdrawal syndrome using a Kratom extract or compounds derived from a Kratom extract. Ethanol addiction results primarily from recreational use where the subject obtains the drug legally but is unable to control their consumption patterns. In one embodiment, the present invention contemplates natural compounds that may provide a reversal of the addiction phenomenon while sparing the patient the clinical manifestations of a classical withdrawal syndrome.

Symptoms of alcohol use include, but are not limited to, slurred speech, lack of coordination, decreased attention span, and/or impaired judgment.

Symptoms of alcohol withdrawal include, but are not limited to, anxiety, shaking (tremors), seizures, increased blood pressure, pulse, and/or temperature, and/or delirium.

Alcoholism (i.e., for example, the chronic, excessive consumption of ethanol) has been recognized as a metabolic disease exhibiting the clinical features of craving for alcohol, loss of control over drinking, tolerance and physical dependence on alcohol, while both epidemiological and experimental studies have demonstrated that genetic factors may be important in determining whether an individual has a high or low vulnerability to develop alcoholism. Evidence also indicates that alcoholism is not characterized by a single gene/single allele inheritance pattern. Instead, multiple genes and environmental factors may interact to increase or decrease an individual's vulnerability to become an alcoholic.

Current research is aimed at investigating whether certain behavioral, physiological and biochemical markers are highly associated with the incidence of alcoholism. One source of these potential biochemical markers include the endogenous opioid system, wherein the opiate system may mediate the reinforcing effects of ethanol. Current research is directed to: (a) the interactions of ethanol with the endogenous opioid system at the molecular level; (b) the existence of genetically determined differences in the response of the endogenous opioid system to ethanol between subjects at high and low risk for excessive ethanol consumption, as well as between lines of animals showing preference or aversion for ethanol solutions; (c) the decrease of alcohol consumption following pretreatment with opioid antagonists; and (d) the possible use of specific opioid receptor antagonists together with behavioral therapy to modify drinking behavior, to control craving and to prevent relapse. Gianoulakis et al., “Genetics of alcoholism: role of the endogenous opioid system” Metab Brain Dis. 9:105-131 (1994).

Some evidence indicates that the endogenous opioid system may play a role in maintaining alcohol drinking behavior. For example, the reinforcing properties of alcohol that lead to continued and repeated bouts of drinking may be due, in part, to alcohol-induced activation of the endogenous opioid system. Blocking the action of endogenous opioid peptides via administration of opioid antagonists significantly attenuates alcohol consumption in animals under a variety of experimental conditions. In clinical trials, opioid receptor antagonists decrease alcohol consumption, relapse rates, subjective high, and alcohol craving in outpatient alcoholics. Opioid receptor antagonists have been proposed to treat alcoholism and alcohol dependence. Froehlich et al., “Opioid involvement in alcohol drinking” Ann NY Acad Sci. 739:156-167 (1994); and Froehlich et al., “Recent developments in alcoholism:opioid peptides” Recent Dev Alcohol. 11:187-205 (1993).

The pleasant effects of alcohol intake may be partially mediated by mu-opiate receptors in the ventral striatum, a central area of the brain reward system. For example, blockade of mu-opiate receptors with naltrexone reduces the relapse risk among some but not all alcoholic individuals. Pronounced alcohol craving may occur among alcoholic individuals with a high availability of mu-opiate receptors in the brain reward system. In one study, the availability of central mu-opiate receptors was measured in vivo with positron emission tomography (PET) and the radioligand carbon 11-labeled carfentanil in the ventral striatum and compared with the severity of alcohol craving as assessed by the Obsessive Compulsive Drinking Scale (OCDS). After 1 to 3 weeks of abstinence, the availability of mu-opiate receptors in the ventral striatum, including the nucleus accumbens, was significantly elevated in alcoholic patients compared with healthy controls and remained elevated when 12 alcoholic patients had these levels measured 5 weeks later (P<0.05 corrected for multiple testing). Higher availability of mu-opiate receptors in this brain area correlated significantly with the intensity of alcohol craving as assessed by the OCDS. These data suggest that abstinent alcoholic patients displayed an increase in mu-opiate receptors in the ventral striatum, including the nucleus accumbens, which correlated with the severity of alcohol craving. Heinz et al., “Correlation of stable elevations in striatal mu-opioid receptor availability in detoxified alcoholic patients with alcohol craving: a positron emission tomography study using carbon 11-labeled carfentanil”. Arch Gen Psychiatry 62:57-64 (2005).

### IV. Cannabinoid Addiction and Withdrawal

In one embodiment, the present invention contemplates a method for treating tetrahydrocannabinol tolerance and/or dependence using a kratom extract. In one embodiment, the method comprises binding at least one component of the kratom extract to a cannabinoid receptor.

Cannabis is believed to be a widely used illicit drug in many western countries. Its psychoactive ingredient, delta-9-tetrahydrocannabinol (THC), produces a variety of effects in animals and humans that are probably mediated by specific cannabinoid receptors in the brain and interactions with several neurotransmitter and neuromodulator systems. For instance, recent research has revealed an important mutual functional relationship between cannabinoids and endogenous opioid systems in mediating the pharmacological and behavioral actions produced by these agents including, but not limited to, drug reinforcement. Perinatal exposure to, and interactions between, cannabinoids and opioids might also have long-term behavioral consequences lasting into adulthood. Further, maternal exposure to THC may affect motivational properties of morphine in male and female adult rats, as measured by an intravenous opiate self-administration paradigm. Ambrosio et al., “The neurobiology of cannabinoid dependence: sex differences and potential interactions between cannabinoid and opioid systems” Life Sci. 65:687-694 (1999).

Advances in cannabis research have paralleled developments in opioid pharmacology whereby a psychoactive plant extract has elucidated novel endogenous signalling systems with therapeutic significance. Cannabinoids (CBs) are chemical compounds derived from Cannabis. The major psychotropic CB, delta-9-tetrahydrocannabinol (Delta(9)-THC), was isolated in 1964 and the first CB receptor (CB(1)R) was cloned in 1990. CB signalling occurs via G-protein-coupled receptors distributed throughout the body. Endocannabinoids are derivatives of arachidonic acid that function in diverse physiological systems. Neuronal CB(1)Rs modulate synaptic transmission and mediate psychoactivity Immune-cell CB(2) receptors (CB(2)R) may down-regulate neuroinflammation and influence cyclooxygenase-dependent pathways. Animal models demonstrate that CBRs play a fundamental role in peripheral, spinal, and supraspinal nociception and that CBs are effective analgesics. Clinical trials of CBs in multiple sclerosis have suggested a benefit in neuropathic pain. However, human studies of CB-mediated analgesia have been limited by study size, heterogeneous patient populations, and subjective outcome measures. Furthermore, CBs have variable pharmacokinetics and can manifest psychotropism. BCs are currently approved as antiemetics in chemotherapy and can be prescribed on a named-patient basis for neuropathic pain. Future selective peripheral CB(1)R and CB(2)R agonists will minimize central psychoactivity and may synergize opioid anti-nociception. Hosking et al., “Therapeutic potential of cannabis in pain medicine” Br J Anaesth. 101:59-68 (2008).

Interactions between opioid and cannabinoid receptors have been studied by epitope tagging mu, delta and kappa opioid receptors with Renilla luciferase and CB1 cannabinoid receptors with yellow fluorescent protein and examined for bioluminescence resonance energy transfer (BRET) signals. Coexpression and receptor-activated interaction of opioid receptors with cannabinoid receptors, was detected by an increase in BRET signal. Further, mu receptor-mediated signaling was attenuated by a CB1 receptor agonist; and this effect is reciprocal in that a CB1 mediated signal was attenuated by a mu receptor agonist. Rios et al., “mu opioid and CB1 cannabinoid receptor interactions: reciprocal inhibition of receptor signaling and neuritogenesis” Br J Pharmacol. 148:385-386 (2006).

Interactions between opioid and cannabinoid systems have been proposed. For example, a modulatory interaction between opioid and cannabinoid systems may exist in the drug reinforcement. Jardinaud et al., “Tolerance to the reinforcing effects of morphine in delta 9-tetrahydrocannabinol treated mice” Behav Brain Res. 173:255-261 (2006). Functionality of the endogenous cannabinoid system undergoes relevant changes in reward-related brain areas in animal models of opiate addiction.

It has been suggested recently that the endocannabinoid system might be a component of the brain reward circuitry and thus play a role not only in cannabinoid tolerance, dependence, and withdrawal, but to other drugs of abuse as well. Changes in endocannabinoid ligands and their receptors have been observed in different brain regions (i.e., for example, those areas related to reinforcement processes) during morphine dependence. Rat brain contents of N-arachidonoylethanolamine (anandamide, AEA, an endocannabinoid) did not change in opiate-dependent animals, despite the presence of physical dependence. By contrast, a significant decrease in the specific binding for CB(1) receptors in the midbrain and the cerebral cortex of morphine-dependent rats was detected. These data suggest altered endocannabinoid transmission during morphine dependence in rats and may be useful in the treatment of morphine addiction. González et al., “Region-dependent changes in endocannabinoid transmission in the brain of morphine-dependent rats” Addict Biol. 8:159-166 (2003).

A relationship between the cannabinoid and opioid receptors in animal models of opioid-induced reinforcement has been reported. The acute administration of a selective central cannabinoid CB1 receptor antagonist (i.e., for example, SR141716A), blocked heroin self-administration in rats, as well as morphine-induced place preference and morphine self-administration in mice. Morphine-dependent animals injected with SR141716A exhibited a partial opiate-like withdrawal syndrome that had limited consequences on operant responses for food and induced place aversion. Additionally, the opioid antagonist naloxone precipitated a mild cannabinoid-like withdrawal syndrome in cannabinoid-dependent rats and blocked cannabinoid self-administration in mice. These results demonstrate a potential cross-interaction between opioid and cannabinoid systems in behavioral responses related to addiction and treatment of opiate dependence. Navarro et al., “Functional interaction between opioid and cannabinoid receptors in drug self-administration” J Neurosci. 21:5344-5350 (2001).

For example, the effects of SR141716A on the rewarding responses of morphine were evaluated in the place conditioning paradigm. SR141716A was able to antagonize the acquisition of morphine-induced conditioned place preference. SR141716A was co-administered with morphine for 5 days, and the withdrawal syndrome was precipitated by naloxone administration. A reduction in the incidence of two main signs of abstinence, wet dog shakes and jumping, was observed. In contrast, an acute injection of the CB(1) antagonist just before naloxone administration was unable to modify the incidence of the behavioural manifestations of the withdrawal, suggesting that only chronic blockade of CB(1) receptors is able to reduce morphine-induced physical dependence. Mas-Nieto et al., “Reduction of opioid dependence by the CB(1) antagonist SR141716A in mice: evaluation of the interest in pharmacotherapy of opioid addiction” Br J Pharmacol. 132:1809-1816 (2001).

By using a limited access heroin self-administration paradigm the cannabinoid CB(1) receptor antagonist N-(piperidin-1-yl)-5-(4-chlorophenyl)-1-(2,4-dichlorophenyl)-4-methyl-1H-pyrazole-3-carboxamide hydrochloride (SR141716A, 0.03-3.0 mg/kg) was shown to suppress heroin self-administration only in opiate-dependent rats but not in non-dependent animals. These results suggest that cannabinoid CB(1) receptor antagonists might be useful in the treatment of opiate addiction. Navarro et al., “Cannabinoid receptor antagonist reduces heroin self-administration only in dependent rats” Eur J Pharmacol. 501:235-237 (2004).

### V. Kratom Opioid Derivatives

Kratom (Mitragynia speciosa korth) is a medicinal herb native to Southeast Asia (i.e, for example, Thailand) used traditionally by laborers to provide increased energy and to treat pain. It has more recently been used by recreational drug users for exploratory purposes. Kratom is legally an unscheduled drug and therefore is legal to own, possess, use, and sell. Recent studies have identified that a large population of individuals with chronic pain who are maintained on prescription opioid analgesics use Kratom as a natural opioid replacement therapy, and has been successfully used in place of methadone, buprenorphine, and other opioid-based treatment regimens. Furthermore, high throughput molecular screening for mitragynine, the predominant alkaloid in Kratom, has found that this molecule binds to central nervous system tissue mu (μ) and kappa (κ)-opiate receptors, α-2-adrenergic receptors, and 5-HT receptors. This data suggests that mitragyinine may have cumulative effects affecting many existing therapeutic approaches including, but not limited to; i) opioid replacement therapies (i.e, for example, methadoneibuprenorphine (at the μ and κ receptors); ii) clonidine therapies (at the α-2-adrenergic receptor); and iii) selective serotonin receptor inhibitor therapies (at 5-HT binding sites).

The concentration of mitragynine in Kratom tree leaves is highly variable and depends upon the phenotype of the tree as well as its location. Kratom was traditionally used as treatment for opium withdrawal, but reports of uncontrolled, compulsive use led to its criminalization in a number of countries, including Australia, Thailand, Malaysia, and Mynamar.^{23, 24} Chronic use of Kratom has been associated with anorexia, weight loss, constipation and hyperpigmentation of the face.²⁵⁻²⁷

In one embodiment, the present invention contemplates a method for treating a subject exposed to opioid analgesic dependence, abuse and addiction. In one embodiment, the opioid dependence, abuse, and addiction is derived from clinical use of a prescription opioid compound. In one embodiment, the opioid dependence, abuse, and addiction is derived from recreational use of an illegal opioid compound. In one embodiment, the method comprises using Kratom (Mitragyna speciosa korth) derived extracts and/or compounds as an opioid replacement therapy to treat addiction to opioids including, but not limited to, oxycodone, fentanyl, hydrocodone, codeine, morphine, hydromorphone, or tramadol.

Much of the information related to Kratom pharmacological effects is derived from user interviews. The clinical effects of Kratom appear to be dose-dependent, where stimulant effects predominate at lower doses, while more traditional opioid sedative effects are more common at higher doses. Effects begin within 5-10 minutes of ingestion and persist for approximately 6 hours. Because it can be ingested in multiple forms (e.g., tea, capsule, or extract, among others), Kratom is a non-stigmatizing, yet an apparently effective, ersatz opioid replacement therapy that can bridge periods of analgesic abuse in highly opioid tolerant individuals.²⁸

In addition to its pharmacologic profile, Kratom has several advantages which further enhance its utility over traditional treatment of opioid addiction, for example; i) Kratom use has never been associated with respiratory depression; ii) Kratom may obviate the morbidity and mortality associated with traditional opioid replacement therapy; iii) cessation from Kratom administration produces symptoms far less pronounced than those induced by cessation from other opioid replacement therapies; and iv) Kratom can be ingested in socially non-stigmatizing forms (i.e., for example, tea) that improve compliance during outpatient treatment of opioid addiction. Nonetheless, the tolerability and efficacy of Kratom in reducing opioid withdrawal in humans has not been clinically evaluated.

Kratom comprises at least twenty (20) indole alkaloids, two of which, mitragynine and 7-hydroxymitragynine, are structurally distinct from opiates.^{2, 3, 35-38} Once isolated and purified, mitragynine binds CNS opioid receptors with high affinity. Using high throughput molecular screening of mitragynine, studies have demonstrated that mitragynine extensively inhibits radioligand binding at several relevant CNS receptor systems. Although mitragynine binds at several CNS receptors, only the dissociation constants for binding at opioid receptors have been determined. These pharmacologic effects appear to be responsible for the observed ability of Kratom to ameliorate and possibly extinguish opioid craving during abstinence.²⁸

In one embodiment, the present invention contemplates a method comprising admninstering Kratom as replacement therapy for an addictive compound, and ceasing Kratom administration under conditions where withdrawal symptoms are reduced and/or prevented.

A. Kratom Extracts

Crystalline products were isolated from the leaves of Mitragyna speciosa that exhibits analgetic and antitussive properties. However, these products had failed to identify the Kratom extracts such as mitragynine for the treatment of opioid receptor based substance abuse. Beckett, “Speciofoline, An Alkaloid from Mitragyna speciosa,” U.S. Pat. No. 3,324,111; and Beckett et al., “Compositions Comprising An Alkaloid of Mitragyna speciosa and Methods of Using Same,” U.S. Pat. No. 3,256,149 (both patents herein incorporated by reference).

Dosage-dependent effects of Kratom extract on animal physiology was recently reported. For example, high doses of Kratom extract decreased the increment of body weight similar to the effect of morphine. Chittrakarn et al., “Inhibitory Effects of Kratom Leaf Extact (Mitragyna speciosa Korth.) on the Rat Gastrointestinal Tract” Journal of Ethnopharmacology 116:173-178 (2007). Anecdotally, there have been suspicions that Kratom has traditionally been used as a substitute for opium when opium is unavailable, or by drug users who are trying to moderate their opium addiction (i.e., for example, self-medication). Opioid substance abuse during chronic alleviation of back pain has been treated using a Kratom derived extract. Boyer et al., Self-Treatment of Opioid Withdrawal Using Kratom, Addiction 103, 1048-1050 (2008).

Kratom components are believed to include, but are not limited to, mitragynine and 7-hydroxymitragynine, that have been reported to agonize the mu-opioid receptor with high affinity. Recent findings suggest that Kratom is purchased from internet sources by some of the 40 million Americans with chronic pain to self-manage opioid withdrawal. Boyer et al., “Self-treatment of opioid withdrawal with a dietary supplement Kratom” Am J Addict 16:352-356 (2007); Yamamoto et al., “Opioid receptor agonistic characteristics of mytragynine pseudoindoxyl in comparison with mitragyine derived from That medicine plant Mytragyna speciosa” Gen Pharmacol 33:73-81 (1999); Thongpradichote et al., “Identification of opioid receptor subtypes in antinociceptive actions of suprasinally administered Mitragynine in mice” Life Sci 62:1371-1378 (1998).

The data presented herein reports on a patient undergoing a protracted use of Kratom for chronic pain treatment and opioid replacement therapy. Boyer et al., “Self-treatment of opioid withdrawal using kratom (Mitragynia speciosa korth)” Addiction 103:1048-1050 (2008). It is currently believed that Kratom is gaining awareness as a ‘natural’ alternative to physician supervised opioid replacement therapy among individuals with chronic pain who are maintained on opioid analgesic agents. Boyer et al., “Self-treatment of opioid withdrawal with a dietary supplement, Kratom” Am J Addict 16:352-356 (2007). In one embodiment, the present invention contemplates a method wherein Kratom attenuates potentially severe opioid withdrawal, yet cessation of Kratom administration itself appears to be associated with modest abstinence symptoms. The pharmacological bases underlying this apparent paradox are uncertain. For example, mitragynine is theorized to stimulate contributions from adrenergic and serotonergic pathways that augment analgesia, but formal binding data have been obtained only for mu-, delta- and kappa opioid receptors. Takayama et al., “Studies on the synthesis and opioid agonistic activities of mitragynine-related indole alkaloids: discovery of opioid agonists structurally different from other opioid ligands. J Med Chem 45:1949-1956 (2002); and Matsumoto et al., “Central antinoceptive effects of mitragynine in mice: contributions from noradrenergic and serotonergic systems” Eur J Pharmacol 317:75-81 (1996).

To delineate the in vitro pharmacology of Kratom more clearly, highthroughput molecular screening of mitragynine activity was conducted at central nervous system receptors (Novascreen Biosciences Corp., Hanover, Md., USA). These studies identified that mitragynine extensively inhibits radioligand binding at several central nervous system receptor systems (See, Table 1).

TABLE 1
Central nervous system receptor binding data for mitragynine.

Percentage inhibition of radioligand binding by mitragynine
at selected receptor systems
Adenosine A2A: 65.66
Adrenergic (Alpha 2): 61.92
Dopamine D2: 54.22
Opioid, mu″ 89.52
Opioid, kappa: 90.21
Opioid, delta: 7.00
Serotonin, 5HT2C: 58.77
Serotonin, 5HT7: 64.41
Dissociation constants for opioid receptor binding
Mu receptor: 204 ± 26 nM
Delta receptor: 2250 ± 120 nM
Kappa receptor: 455 ± 47 nM

The clinical implication of these results is that mu-opioid agonism may avert withdrawal symptoms, while kappa agonism attenuates reinforcement and produces aversion. Narita M. et al., “Regulations of opioid dependence by opioid receptor types” Pharmacol Ther 89:1-15 (2001). In addition, mitragynine, through its putative alpha-2 adrenergic agonist activity, may mimic adjunctive therapies for opioid withdrawal such as clonidine. Mitragynine, therefore, may exert several convergent pharmacological effects that could attenuate opioid withdrawal symptoms and blunt cravings.

Adverse effects induced by Kratom administration are poorly described. For example, respiratory depression, coma, pulmonary edema and death resulting from Kratom administration have not been reported despite an agonistic stimulation of mu-opioid receptors mediated by mitragynines. Furthermore, the protracted use of Kratom as a single therapy did not appear to produce any significant adverse effects in this patient; not until co-administration with modafinil was a potential adverse effect of Kratom identified. The exact mechanisms that contribute to seizure are undefined. The mitragynines, their metabolites or other components of Kratom could potentially exhibit proconvulsant properties similar to atypical opioids such as tramadol, the meperidine metabolite normeperidine and propoxyphene. Wills et al., “Drug- and toxin-associated seizures” Med Clin North Am 89: 1297-1321 (2005). Synergism between Kratom and modafinil might also produce seizure, but considering that modafinil is not likely to possess proconvulsant properties, this latter mechanism appears speculative. The relative risk/benefit ratio resulting from long-term Kratom administration is presently unknown.

Nonetheless, current trends of Kratom use may reflect an increasing public interest in obtaining alternative therapies for chronic medical problems. Wills et al., “Drug- and toxin-associated seizures” Med Clin North Am 89:1297-1321 (2005); Eisenberg et al., “Unconventional medicine in the United States. Prevalence, costs, and patterns of use” N Engl J Med 328:246-252 (1993). One reason for this increased interest is that some patients with chronic pain may misunderstand that formal drug treatment is reserved for users of illegal substances. Another reason is that many existing systems that treat chronic pain or problematic opioid use frequently under-prescribe analgesics, require treatment contracts, demand ongoing drug testing and/or otherwise stigmatize those who seek care. These disadvantages of current approaches to substance abuse treatment result in patient resistance to seek physician advice and treatment. Joranson et al., “Pain management, controlled substances, and state medical board policy: a decade of change” J Pain Symptom Manag 23:138-147 (2002).

Toxicity studies of lyophilized Kratom extraction into water have failed to produce respiratory depression.³⁴ Toxicity studies using mitragynine performed in the rat failed to identify respiratory depression, even at doses of 800 mg/kg administered via intraperitoneal dosing.²⁹

Direct observation by addiction medicine experts of former injection drug abusers who abruptly ceased Kratom has revealed an abstinence syndrome with symptoms including, but not limited to, rhinorrhea, insomnia, lacrimation, lethargy, myalgias, or myoclonus.²⁷ Kratom withdrawal has not been associated with other common opiate withdrawal symptoms such as, gastrointestinal disturbances or severe malaise. In one embodiment, the present invention contemplates a method comprising administering Kratom to treat and/or prevent an opiate withdrawal syndrome following cessation of opiate use by a subject suffering from addiction and/or substance abuse.

In one embodiment, the present invention contemplates a method comprising self-treating a subject suffering from chronic pain with a Kratom extract. In one embodiment, the Kratom extract treatment does not develop tolerance in the subject. In one embodiment, the tolerance comprises functional tolerance. In one embodiment, the Kratom extract treatment does not develop a Kratom addiction in the subject.

B. Mitragynine

Mitragynine has been reported as a major alkaloidal component in Mitragyna speciosa. Other work has indicated that mitragynine exhibits analgesic activity mediated by opioid receptors. By utilizing this natural product as a lead compound, synthesis of some derivatives, evaluations of the structure-activity relationship, and surveys of the intrinsic activities and potencies on opioid receptors were performed with guinea pig ileum. For example, oxidative derivatives of mitragynine, (i.e., for example, mitragynine pseudoindoxyl and 7-hydroxymitragynine) were found to be opioid agonists with higher potency than morphine. Takayama et al., “Studies on the Synthesis and Opioid Agonistic Activities of Mitragynine-Related Indole Alkaloids: Discovery of Opioid Agonists Structurally Different from Other Opioid Ligands” Journal of Medicinal Chemistry 45:1949-1956 (2002). Further studies identified high affinity binding properties coupled with opioid antagonistic effects of mitragynine derivatives on individual opioid receptors, including, but not limited to, the μ-, δ- and κ-opiate receptors. Mitragynine and derivatives appear to bind to α₂-adrenergic as well as serotonergic receptors in the CNS. Kratom-derived compounds therefore integrate with the pharmacology of established treatments for opioid withdrawal such as methadone, buprenorphine, and clonidine. Importantly, the literature contains no reports of coma, respiratory depression or death from Kratom overdose.

It was reported that potent opioid agonistic activities of mitragynine and its analogues were found in in vitro and in vivo experiments during studies of mechanisms underlying the analgesic activity. Takayama, “Chemistry and Pharmacology of Analgesic Indole Alkaloids from the Rubiaceous Plant, Mitragyna speciosa” Chemical and Pharmaceutical Bulletin 52:916-928 (2004). Mitragynine structural features which differ from those of morphine were elucidated by pharmacological evaluation of the natural and synthetic derivatives. Among the mitragynine derivatives, 7-hydroxymitragynine, a minor constituent of Mitragyna speciosa, was found to exhibit potent antinociceptive activity in mice.

Mitragynine has also been reported as a partial opioid agonist producing similar effects to morphine. Alternatively, the mitragynine derivative, 7-hydroxymitragynine, has been reported to be more potent than morphine. Although it is not necessary to understand the mechanism of an invention, it is believed that mitragynine and 7-hydroxymitragyinie may activate supraspinal mu- and delta-opioid receptors, thereby explaining their use by chronic narcotics users to ameliorate opioid withdrawal symptoms”. Babu et al., “Opioid Receptors and Legal Highs: Salvia divinorum and Kratom” Clinical Toxicology 46:146-152 (2008).

Pharmacological studies on mitragynine, the predominant alkaloid of Kratom, were first published in 1972.²⁹ Researchers who sought a novel analgesic with less abuse liability than the phenanthrene opioids conducted a battery of animal studies to investigate the analgesic potential and opioid actions of mitragynine. These studies demonstrated analgesic and antitussive properties comparable to codeine. Unlike codeine, mitragynine was not blocked by nalorphine and had much less respiratory depression. Further, mitragynine suppressed the opioid withdrawal syndrome. Moreover, mitragynine was active only via the oral and intraperitoneal routes of administration (in an equal ratio) but was inactive via the parenteral routes, a feature that diminished its abuse liability. The analgesic activity of mitragynine was again investigated in the tail-pinch and hot-plate tests resulting in antinociceptive activity that was completed abolished by naloxone, a pure opioid receptor antagonist. This indicated the involvement of supraspinal opioid receptors in the analgesic actions of mitragynine and a renewed interest in the pharmacology of this molecule.³⁰

Mitragynine, in a manner similar to morphine, is believed to stimulate descending noradrenergic and serotonergic systems to produce analgesia.^{30, 31} For example, the α₂-adrenoceptor antagonist, idazoxan, and the 5-HT receptor antagonist, cyproheptadine, antagonized the analgesic effects of mitragynine. This work indicated that mitragynine may stimulate the release of endogenous norepinephrine and serotonin, similar to the actions of other opioid ligands.^{30, 31} In a companion study, the inhibition of electrically stimulated contraction in the guinea-pig ileum is reversed by naloxone, with the involvement of mu- and delta-opioid receptors identified through the use of subtype selective antagonists.³²

The binding affinities for mitragynine at the three opioid receptors were determined using guinea pig brain membranes. This data indicated that mitragynine is a mu-opioid selective opioid ligand with a pKi value of 8.14±0.28 and a relative affinity of 88.7% for the mu- over the delta- and kappa-opioid receptors. The pKi values at the delta- and kappa-opioid receptor were 7.22±0.21 and 5.96±0.22, respectively. ³³

### VI. Sphingolipids

In one embodiment, the present invention contemplates a composition comprising a kratom extract and a plurality of sphingolipids. In one embodiment, the sphingolipids comprise plant sphingolipids. In another embodiment, the present invention contemplates a method of reducing pain using a kratom extract comprising a plurality of sphingolipids, wherein the kratom sphingolipids increase the level of analgesia in comparison to a biomodal opioid agonist. In one embodiment, the sphingolipids comprise plant sphingolipids.

In one embodiment, the present invention contemplates a composition comprising mitragyinine and at least on sphingolipid. In one embodiment, the sphingolipid comprises a plant sphingolipid. In one embodiment, the present invention contemplates a method of reducing pain using mitragynine and at least one sphingolipid, wherein the sphingolipids increase the level of analgesia in comparison to mitragynine alone. In one embodiment, the sphingolipid comprises a plant sphingolipid.

A. Plant Sphingolipids

Plant sphingolipids comprise structural features differing from those found in animals and fungi. Sphingolipid modifications are found in plants and recent advances in the functional characterization of genes is gaining new insight into plant sphingolipid biosynthesis. Recent studies indicate that plant sphingolipids may be also involved in signal transduction, membrane stability, host-pathogen interactions and stress responses. Sperling et al., “Plant sphingolipids: structural diversity, biosynthesis, first genes and functions” Biochimica et Biophysica Acta 1632:1-15 (2003)

1. Introduction

Sphingolipids are ubiquitous membrane components in eukaryotic cells and in a few bacteria. K. A. Karlsson, Lipids 5 (1970) 878-891; and K. A. Karlsson, Chem. Phys. Lipids 5 (1970) 6-43. Their chemical structure differs from the more commonly known glycerolipids in having a ceramide backbone, which consists of a fatty acid attached to a long-chain amino alcohol. Recent interest is focusing on the role of sphingolipids in serving as intra- and intercellular second messengers regulating cell growth, differentiation, apoptosis, and pathogenic defense. van Meer et al., Biochim. Biophys. Acta 1486 (2000) 145-170; and Dickson et al., Biochim. Biophys. Acta 1583 (2002) 13-25.

Compared to the tremendous research on bioactive sphingolipids in mammalian systems and Saccharomyces cerevisiae published during the last two decades, there is a paucity of studies using plant systems. Studies on sphingolipid metabolism in plants have focused on demonstrating and characterizing the in vitro activities of enzymatic steps in major pathways. D. V. Lynch, Methods Enzymol. 311 (2000) 130-149. The success in elucidating additional aspects of their metabolism and in recognizing functions are mainly due to the fact that genes controlling biosynthetic steps of sphingolipids have been identified only recently from plants and some other phyla.

In many studies, S. cerevisiae served as a model organism to study sphingolipid metabolism and may be the first eukaryotic organism in which all sphingolipid metabolic genes are identified. However, these data do not apply to the biosynthesis and functions of the structural diverse plant sphingolipids. For example, divergencies in the biosynthetic pathway of plants lead to cerebrosides and glycosyl inositol phosphorylceramides (GIPC) with a preference for D8-unsaturated long-chain bases (LCB) not present in baker's yeast. Imai et al., Biosci. Biotechnol. Biochem. 61 (1997) 351-353; Kawaguchi et al., Biosci. Biotechnol. Biochem. 64 (2000) 1271-1273; Imai et al., J. Plant Physiol. 157 (2000) 453-456; Mano et al., Biosci. Biotechnol. Biochem. 63 (1999) 619-626; Fujino et al., J. Cereal Sci. 1 (1983) 159-168; Ohnishi et al., Biochim. Biophys. Acta 752 (1983) 416-422; W. R. Morrison, Chem. Phys. Lipids 11 (1973) 99-102. Because cerebrosides and unsaturated LCB are absent in yeast, this organism has been used as an expression platform for heterologous genes involved in plant sphingolipid synthesis.

In this way, genes encoding enzymes modifying the ceramide core have been recently identified from a variety of different phyla including plants. Sperling et al., J. Biol. Chem. 273 (1998) 28590-28596; Sperling et al., FEBS Lett. 494 (2001) 90-94; Leipelt et al., J. Biol. Chem. 276 (2001) 33621-33629; Terries et al., J. Biol. Chem. 277 (2002) 25512-25518; and Mitchell et al., J. Biol. Chem. 272 (1997) 28281-28288. Sequence comparisons provide evolutionary relationships of some of these proteins. Sperling et al., Prostaglandins Leukot. Essent. Fat. Acids 68 (2003) 73-95. An extrapolation of the success with S. cerevisiae as a model suggests that the generation of plant mutants affecting sphingolipid metabolism will promote the elucidation of sphingolipid functions. For example, targeted gene disruption by homologous recombination established for: i) the moss Physcomitrella patens (Schafer et al., Plant J. 11 (1997) 1195-1206; and Girke et al., Plant J. 15 (1998) 39-48; ii) RNAi antisense inactivation (Mourrain et al., Genet. Eng. (New York) 22 (2000) 155-170; Wesley et al., Plant J. 27 (2001) 581-590); and iii) the identification of transposon-tagged Arabidopsis thaliana mutants (Bouche et al., Curr. Opin. Plant Biol. 4 (2001) 111-117; and Thorneycroft et al., J. Exp. Bot. 52 (2001) 1593-1601), may provide opportunities to identify and characterize new functions for plant sphingolipids. Structures of sphingolipids found in plants, comprise seemingly minor structural modifications as compared to other phyla which may have unexpected relevance on sphingolipid functions.

2. Structural Diversity of Plant Sphingolipids

Sphingolipids are commonly generated by the addition of a polar head group to ceramides which in turn are composed of a 2-amino-1,3-dihydroxyalkane (LCB) moiety bonded to an N-acylated fatty acid (i.e., for example, comprising 14-26 carbon atoms). Complex sphingolipids, such as cerebrosides and GIPC (phytoglycolipids) may be formed by the addition of various glycosyl residues and other polar phosphate-containing headgroups to the ceramide. Depending on the source, this basic ceramide structure can be modified by differences in chain length, methyl branching, insertion of additional hydroxy groups, and degree of unsaturation.

2.1. Long-Chain Bases

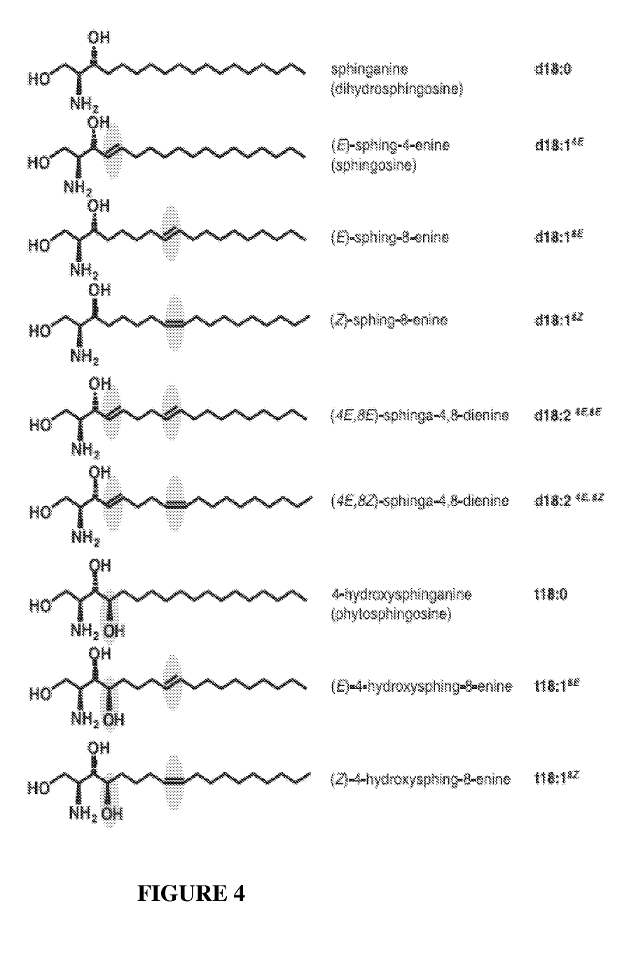

In mammals, the LCB moiety is mostly (E)-sphing-4-enine (sphingosine, d18:1⁴), whereas in the yeast S. cerevisiae, the predominant LCB is 4-hydroxysphinganine (phytosphinganine, t18:0) formed by the desaturation or hydroxylation of sphinganine (d18:0) at C-4, respectively. In contrast, the sphingoid base composition of plants is more variable, being composed of up to eight different C18-sphingoid bases derived from D-erythro-sphinganine. See, FIG. 4. Due to an additional cis- or trans-desaturation at C-8, the predominating regioisomers of unsaturated plant LCB are (E/Z)-sphing-8-enine (d18:1⁸), (4E,8E/Z)-sphinga-4,8-dienine (d18:2^{4,8}) and (8E/Z)-4-hydroxy-8-sphingenine (t18:1⁸), whereas d18:1⁴ is virtually absent and d18:0 and t18:0 are only present in minor proportions. Other LCB differing in chain length are present as minor components in plant sphingolipids.

In Euphorbia characias, as well as in several other organisms, saturated and D6-(Z)-unsaturated tetrahydroxysphingenine derivatives may occur, suggesting the presence of an additional C5-LCB hydroxylase. Rupcic et al., Chem. Phys. Lipids 91 (1998) 153-161; and Li et al., Tetrahedron Lett. 36 (1995) 3875-3876. The occurrence of a D6-(Z)-double bond may be either due to the activity of an “exotic” LCB desaturase or to a serine palmitoyltransferase accepting D4-(Z)-myristoyl-CoA. The occurrence of D8-unsaturated LCB is not restricted to plants. For example, S. cerevisiae contain a di-unsaturated, methyl-branched LCB, (4E,8E)-9-methylsphinga-4,8-dienine in their cerebrosides. Toledo et al., Biochemistry 38 (1999) 7294-7306; Sakaki et al., Yeast 18 (2001) 679-695; Ohnishi et al., J. Jpn. Oil Chem. Soc. 45 (1996) 51-56; Abe et al., Biosci. Biotechnol. Biochem. 58 (1994) 1671-1674. In contrast, t18:0 is the predominating LCB in GIPC. Lester et al., Adv. Lipid Res. 26 (1993) 253-274; Jennemann et al., Eur. J. Biochem. 259 (1999) 331-338; Jennemann et al., Eur. J. Biochem. 268 (2001) 1190-1205.

As known so far, the occurrence of both D8 cis/transisomers seems to be restricted to plant sphingolipids. Ratios of D8-trans- to D8-cis-isomers varying from 91:9 in cucumber to 4:86 in wheat have been found in the leaf cerebrosides of different plant species. In A. thaliana, the (8Z)-t18:1 is the most abundant LCB in leaf cerebrosides recovered from lipid extracts, whereas direct alkaline hydrolysis of whole leaves indicates that the (8E)-t18:1 isomer is most abundant. Taking into account that GIPC are hardly extractable in organic solvents (i.e., for example, chloroform/methanol) which are suitable for the extraction of most membrane lipids including cerebrosides, these data suggest that other complex sphingolipids such as GIPC must be more abundant than monoglucosylceramides in A. thaliana leaves. Z. Imre, Z Naturforsch. 29c (1974) 195-200. The analysis of these complex phytoglycolipids is handicapped by complicated extraction and purification procedures and only a few plant GIPC have been analysed in detail. Carter et al., Biochemistry 8 (1969) 383-388; Kaul et al., Plant Physiol. 55 (1975) 120-129; Kaul et al., Biochemistry 17 (1978) 3569-3575; Hsieh et al., Biochemistry 17 (1978) 3575-3581; Hsieh et al., J. Biol. Chem. 256 (1981) 7747-7755; and Laine et al., Methods Enzymol. 138 (1987) 186-195. Therefore, at present, no exact data on the quantitative proportions of cerebrosides and GIPC in plants are available.

A comparison of the LCB compositions of A. thaliana leaves derived from lipid extracts (i.e., for example, a cerebroside fraction) and from lipid-depleted tissues (i.e., for example, a GIPC fraction) are pointing to a predominance of GIPC compared to cerebrosides and further suggests that there is a channeling of the (8Z)-t18:1 into cerebrosides and of the (8E)-isomer into GIPC. Furthermore, the relative proportions of di- and trihydroxybases in cerebrosides differ with plant species as well, for example, from 78% dihydroxybases in soybean to 87% trihydroxybases in A. thaliana, whereas the glucosylceramides of leaf and root tissues have similar LCB compositions. These studies suggest that plants maintain two separate ceramide pools for the biosynthesis of cerebrosides and GIPC, which could be achieved by different ceramide selectivities of glucosylceramide synthase (GCS) and inositol phosphorylceramide synthase or by restricting access of the enzymes to spatially separated substrate pools.

2.2. Fatty Acyl Amides

In the ceramide backbones of plant sphingolipids, more than 10 different fatty acids can be N-acylated to the eight different LCB mentioned above. These fatty acids are almost exclusively α-D-hydroxylated and vary in their chain lengths from C₁₄ to C₂₆, including chains of odd carbon numbers. Imai et al., Biosci. Biotechnol Biochem. 59 (1995) 1309-1313; Imai et al., Lipids 35 (2000) 233-236; and Bohn et al., Arch. Biochem. Biophys. 387 (2001) 35-40. Saturated C₁₆, C₂₀, C₂₂ and C₂₄ α-hydroxylated fatty acids are most abundant, whereas ω9-monounsaturated very long-chain fatty acids ranging from C₂₂ to C₂₆ occur in low proportions. The occurrence of 2-hydroxy nervonic acid (24h:1) is characteristic for the leaf cerebrosides of some chillingresistant cereals. Imai et al., In: J. P. Williams, M. U. Khan, N. W. Lem (Eds.), Physiology, Biochemistry and Molecular Biology of Plant Lipids, Kluwer Academic Publishing, Dordrecht, 1997, pp. 224-226.

The occurrence of ω9-cis-unsaturated very long-chain fatty acyl amide residues in sphingolipids may be attributed to the sequential fatty acid elongation of oleoyl-CoA resulting in a series of ω9-monounsaturated very long-chain fatty acyl-CoA of 22-26 carbons which could serve as substrate for the plant ceramide synthase. In free ceramides, non-hydroxylated fatty acids can account for 1-32%, whereas in leaf cerebrosides, they are minor constituents ranging from 1% to 3%. Cahoon et al., Plant Physiol. 95 (1991) 58-68; and Ohnishi et al., Agric. Biol. Chem. 46 (1982) 2855-2856. In several plants, even 2-3% of 2,3-dihydroxy fatty acids have been detected, suggesting the existence of a regio-unselective acyl amide α-hydroxylase or of a C3-hydroxylase. Ito et al., Agric. Biol. Chem. 49 (1985) 539-540; and Ohnishi et al., Agric. Biol. Chem. 49 (1985) 3327-3329.

In yeast GIPC, fatty acids of 26 carbons in length are predominating, the majority of which is hydroxylated at the α-position. Lester et al., J. Biol. Chem 268 (1993) 845-856; and Nurminen et al., Biochem. J. 125 (1971) 963-969. The presence of 2,3-dihydroxy acids has been described in N-acyl-4-hydroxysphinganine isolated from S. cerevisiae, whereas a Δ3-(E)-unsaturation of 2-hydroxy fatty acids appears to be a modification restricted to some fungal cerebrosides. Prostenik et al., Lipids 8 (1973) 325-326; Weinert et al., Chem. Phys. Lipids 11 (1973) 83-88; Fujino et al., Biochim. Biophys. Acta 486 (1976) 161-171; and Ballio et al., Biochim. Biophys. Acta 573 (1979) 51-60.

These data indicate not only the presence of an acyl amide C3-hydroxylase in plant and baker's yeast, but also of an acyl amide Δ3-(E)-desaturase in some pathogenic fungi. The corresponding genes coding for these modifications at C-3 have not been identified.

2.3. Polar Head Group

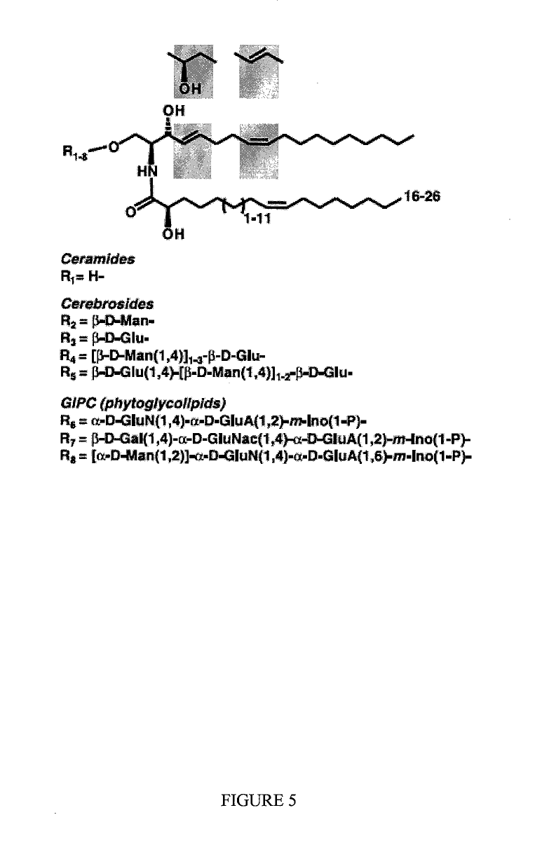

Studies on the molecular species of ceramide residues show that almost all possible combinations of LCB and fatty acids occur in nature giving rise to two types of complex plant sphingolipids. See, FIG. 5. In plants, neutral cerebrosides carry one to four glycosyl residues attached to the primary hydroxyl group of the sphinganine derivatives, whereas in the negatively charged GIPC (phytoglycolipids), inositol-1-phosphate is linked as a phosphodiester to C-1 of the ceramide backbone, which may be further extended by oligosaccharide chains. GPIC core structure nor biosynthesis analyses has been studied in detail in plants.

There are at least two different monoglycosyl ceramides in plants carrying either β-D-mannosyl- or β-D-glucosyl residues. The glucosylceramide is mainly used for further β(1→4) linked mannosylations resulting in series of di-, tri- and tetraglycosyl ceramides, which are terminally capped by a glucosyl residue apparently preventing further chain elongation. Fujino et al., Proc. Jpn. Acad. 58B (1982) 36-39; and Fujino et al., Agric. Biol. Chem. 49 (1985) 2753-2762. Therefore, it seems unlikely that the cellobiosyl ceramide may act as a primer for cellulose synthesis as proposed for β-sitosterol glucosides in plants. Peng et al., Science 295 (2002) 147-150. More than 20 different glucosylceramide species with 12 species comprising each more than 1 mol % of the total cerebroside mixture have been determined in some plant species. For example, galactosyl ceramides (i.e, for example, neuraminic (sialic) acid containing ceramides (e.g., gangliosides)) and sphingomyelin, all of which are typical mammalian sphingolipids, have not been found in higher plants. Huwiler et al., Biochim. Biophys. Acta 1485 (2000) 63-99. In plants, the glucosylceramides typically account for less than 5 mol % of the total lipids, but are quantitatively important components of the outer (apoplastic) monolayer of the plasma membrane comprising 7-30 mol % of membrane lipids. Lynch et al., In: J.-C. Kader, P. Mazliak (Eds.), Plant Lipid Metabolism, Kluwer Academic Publishing, Dordrecht, 1995, pp. 239-241. On the other hand, the bilayer distribution of sphingolipids in the tonoplast has not been determined yet. The same is true for the intracellular location and transbilayer distribution of GIPC in plants. The sum of both, cerebrosides and GIPC, may be significantly higher than anticipated. If they are concentrated in one leaflet of bilayer membranes, the proportion of phospholipids would be significantly reduced.

3. Characterization of Plant Genes for Sphingolipid Biosynthesis

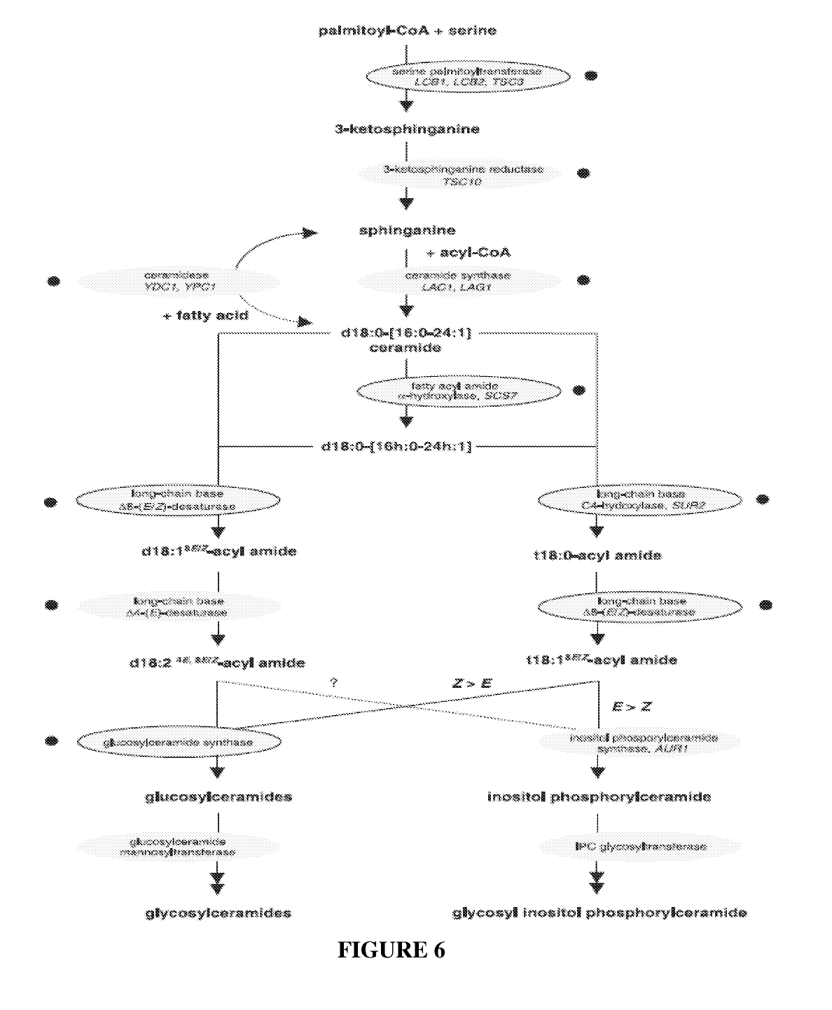

A potential pathway for sphingolipid synthesis in plants has been proposed. See, FIG. 6. The identified orthologous plant genes are indicated by black boxed enzyme names. Sequences identified in the genome of A. thaliana are included and are marked by black dots.

3.1. Ceramide Synthesis

Sphingolipid biosynthesis starts with the condensation of acyl-CoA (mainly palmitoyl-CoA) and L-serine to yield 3-ketosphinganine, catalyzed by the palmitoyl-CoA:L-serine C-palmitoyltransferase (EC 2.3.1.50). Detection of this enzyme activity in plant microsomes by in vitro assays points to a localization in the endoplasmic reticulum (ER). The reaction can be specifically inhibited by L-cycloserine, β-chloro-L-alanine and by the antifungal agents sphingofungin B and C. Zweerink et al., J. Biol. Chem. 267 (1992) 25032-25038. As shown for the yeast enzyme, the serine palmitoyltransferase from plants may also consist of two essential subunits, Lcb1 and Lcb2. Gable et al., J. Biol. Chem. 275 (2000) 7597-7603.

An LCB2 cDNA from A. thaliana has been functionally expressed in a yeast mutant defective in serine palmitoyltransferase activity. Tamura et al., Plant Cell Physiol. 42 (2001) 1274-1281. Expression of a green-fluorescent protein fusion product in tobacco cells showed that Lcb2 is localized in the endoplasmic reticulum (ER). Inspection of the complete Arabidopsis genome database suggests a second hypothetical LCB2-like gene (AB074928) and a putative LCB1-like gene (AB063254). The serine palmitoyltransferase has a strong preference for palmitoyl-CoA, but also palmitelaidoyl-CoA with a trans-double bond at C-9, was still an effective substrate. Saturated acyl-CoAs of shorter or longer chain length as well as palmitoleoyl-CoA with a cis-double bond at C-9 were highly discriminated. Lynch et al., Plant Physiol. 103 (1993) 1421-1429. The specificity of the plant serine palmitoyltransferase for unsaturated C₁₈ acyl-CoA such as oleic-, linoleic- or linolenic-CoA representing the main fatty acyl residues in plants, has not been investigated.

It has been shown, that very long-chain polyunsaturated fatty acids such as eicosapentaenoic acid (20:5) produced by the fungus Mortierella alpina are not incorporated into its cerebrosides. Batrakov et al., Chem. Phys. Lipids 117 (2002) 45-51. In a second step, 3-ketosphinganine is reduced with NADPH by the D-erythro-sphinganine:NADP⁺ 3-oxidoreductase (EC 1.1.1.102) to yield sphinganine (D-erythro-2-amino-1,3-dihydroxyalkane). The TSC10 gene encoding this membrane-bound and essential enzyme in S. cerevisiae has been located at the cytosolic side of the ER in mammalian cells. Beeler et al., J. Biol. Chem. 273 (1998) 30688-30694; and Mandon et al., J. Biol. Chem. 267 (1992) 11144-11148.

Two homologs encoding putative 3-ketosphinganine reductases (Accession Numbers NM_{—}111481 and NM_{—}121925) can be found in the A. thaliana genome, but their functions have not been identified. In the next step, the amino group of sphinganine is acylated to yield ceramide (N-acyl sphinganine). In yeast, this reaction is catalysed by two similar acyl-CoA:sphinganine N-acyltransferases (ceramide synthases, EC 2.3.1.24), Lac1 and Lag1, requiring long chain acyl-CoA. Schorling et al., Mol. Biol. Cell 12 (2001) 3417-3427; and Guillas et al., EMBO J. 20 (2001) 2655-2665. Two cDNA homologs of LAG1 (AF198179, AF198180) may code for two putative sphinganine N-acyltransferases in A. thaliana. Brandwagt et al., Proc. Natl. Acad. Sci. U.S.A. 97 (2000) 4961-4966. The activity of such an enzyme has been demonstrated in microsomal membranes of squash, bean and corn, suggesting a localization in ER membranes.

D-Erythro-sphinganine and -sphingosine serve as substrates for the N-acyltransferase, whereas DL-threo-sphinganine and 4-hydroxysphinganine do not. In yeast, the ceramide synthases obviously channel C₂₆ fatty acids into GIPC. In plants, the distribution of hydroxy fatty acyl chains in glucosylceramides is paralleled by the substrate specificity of the enzymes using C₁₆ to C₂₄ acyl-CoA. These data suggest a role for this enzyme in determining the acyl amide compositions of both cerebrosides and GIPC. Hydroxy acyl chains do not function as substrate, indicating that α-hydroxylation apparently occurs following ceramide formation.

The second mechanism for ceramide synthesis utilizing predominantly free palmitic acid in plants has been demonstrated in vitro. Ceramidase activity is stimulated by the addition of erythro-sphinganine, whereas (E)-sphing-4-enine is a poor substrate and 4-hydroxysphinganine inhibits ceramide formation. In yeast, two ceramidases preferring either N-acyl sphinganine, Ydc1, or N-acyl 4-hydroxysphinganine, Ypc1, have been cloned. From these two enzymes, Ypc1 is probably involved in sphingolipid degradation, showing acyl-CoA-independent reverse activity in ceramide formation. Mao et al., J. Biol. Chem. 275 (2000) 6876-6884; and Mao et al., J. Biol. Chem. 275 (2000) 31369-31378. A putative ceramidase is also present in A. thaliana, but the YPC1 gene (BAB60897) has not been cloned. Comparison of the in vitro activities of the sphinganine N-acyltransferase and the reverse ceramidase in plant membrane preparations indicates that ceramide formation in vivo may occur predominantly by the acyl-CoA dependent reaction. The role of the reverse ceramidase in vivo remains unclear, though it may act as salvage mechanism for otherwise cytotoxic free fatty acids and LCB.

3.2. Modifications of the Hydrophobic Ceramide Core

Once the ceramide backbone is established, the LCB and acyl amide residues are further modified by desaturations and/or hydroxylations to form the molecular species commonly found in plants. Only a few enzymatic activities required for these modifications have been demonstrated in vitro, and the true substrates for these reactions, that is, free LCB, ceramide, cerebroside or GIPC, are still not known with certainty.

3.2.1. C4-LCB Hydroxylation

In plants, sphinganine can be either desaturated to (E)-sphing-4-enine or it can be C₄-hydroxylated to yield 4-hydroxysphinganine (phytosphinganine), most of which is further desaturated to yield cis/trans-isomers of Δ8-unsaturated LCB. Laine et al., Biochemistry 12 (1973) 1106-1111. In S. cerevisiae, lacking Δ4-LCB desaturation, a non-essential SUR2/SYR2 gene responsible for C4-LCB hydroxylation to give C₁₈- and C₂₀-phytosphinganine has been identified by gene disruption. Haak et al., J. Biol. Chem. 272 (1997) 29704-29710; and Grilley et al., J. Biol. Chem. 273 (1998) 11062-11068. It is unclear whether sphinganine, N-acyl-sphinganine (dihydroceramide) or both are substrates for hydroxylation at C-4. SUR2-orthologous sequences have been found in Schizosaccharomyces pombe, Candida albicans and A. thaliana. Heterologous expression of each of two Sur2-like genes identified from A. thaliana in a sur2Δ-null mutant lacking C4-LCB-hydroxylation resulted in the formation of D-ribo-C₁₈- and —C₂₀-phytosphinganine indicating the presence of two isoenzymes for C4-LCB hydroxylation.

3.2.2. Δ4-LCB Desaturation

Plants are believed to synthesize sphingolipids with Δ4-transunsaturated LCB. Laine et al., Biochemistry 12 (1973) 1106-1111. In mammals, the Δ4-(E)-desaturation occurs at the cytosolic face of ER membranes at the level of N-acyl sphinganine. Causeret et al., Lipids 35 (2000) 1117-1125. NADH or NADPH and molecular oxygen are required as co-factors, whereas cyanide, divalent copper, dithiothreitol and antibodies raised against cytochrome b5 inhibit sphingolipid Δ4-(E)-desaturase (dihydroceramide desaturase) activity. Factors that influence the mammalian enzyme activity include the alkyl chain length of the LCB (C₁₈>C₁₂>C₈), the acyl amide chain (C₈>C₁₈), the stereochemistry of the LCB (D-erythro>L-threo-N-acyl sphinganines) and the nature of the headgroup with highest activity observed with N-acyl sphinganine, some with dihydrosphingomyelin, but no activity with free C₁₈-sphinganine or dihydroglucosylceramide. Michel et al., J. Biol. Chem. 272 (1997) 22432-22437.

Plant orthologous Δ4-(E)-desaturase sequences have been found in A. thaliana (AF220201) and Lycopersicon esculentum. Both the sphingolipid Δ4-(E)-desaturase as well as the SUR2-like C4-hydroxylase sequences show three conserved histidine motifs characterizing all membrane-bound fatty acyl desaturases. Shanklin et al., Annu. Rev. Plant Physiol. Plant Mol. Biol. 49 (1998) 611-641. But both sequences do not contain a cytochrome b5 domain, which inter alia has been found in sphingolipid Δ8-desaturases and some acyl amide α-hydroxylases. Sperling et al., Eur. J. Lipid Sci. Technol. 103 (2001) 158-180.

3.2.3. Δ8-LCB Desaturation

Like Δ4-(E)-desaturation, Δ8-unsaturated sphingolipids do not occur in baker's yeast. Interestingly, only the transisomers of Δ8-unsaturated LCB have been found in Pichia pastoris, Rhynchosporium secalis, M. alpina and other fungi, whereas a mixture of cis- and trans-isomers is characteristic for plants. For example, genes encoding plant sphingolipid Δ8-desaturases (EC 1.14.99) have been functionally identified from A. thaliana, Brassica napus, Helianthus annuus and Borago officinalis. Sperling et al., Biochem. Soc. Trans 28 (2000) 638-641; Sperling et al., Arch. Biochem. Biophys. 388 (2001) 293-298. Surprisingly, heterologous expression of these cDNA sequences in S. cerevisiae resulted in significant proportions of both cis- and transisomers of plant characteristic 4-hydroxysphing-8-enines not present in wild-type yeast cells.

The presence of C₁₈- and C₂₀-(8E/Z)-4-hydroxysphing-8-enines in these transgenic cells can be ascribed to the activity of a stereounselective sphingolipid Δ8-desaturase lacking absolute chain length specificity. Depending on the plant source, different and characteristic E/Z-ratios ranging from 3:1 to 7:1 were obtained when using the same yeast expression system. Any influence from an unspecific yeast isomerase could be excluded because wild-type yeast incorporating exogenously applied, synthetic Δ6-(E)-hexadecenoic acid into sphingolipids yielded exclusively (E)-4-hydroxyshing-8-enine and do not show any conversion to the (Z)-isomer. The formation of both (E)- and (Z)-double bonds results from a syn-elimination of two vicinal hydrogen atoms from two different substrate conformers. Beckmann et al., Angew. Chem. Int. Ed. Engl. 41 (2002) 2298-2300. Low but distinct kinetic isotopic effects suggest a preferential attack at C-8 of 4-hydroxysphinganine with anti-orientation en route to the E-isomer and at C-9 with gauche-orientation to the Z-isomer. Since both isomers are generated by the same enzyme, a uniform mechanism involving a transient C-centered radical can be proposed. Therefore, the sphingolipid Δ8-desaturase is different from the hitherto studied and stereospecifically operating fatty acyl (Z)-desaturases including the sphingolipid Δ4-(E)-desaturase from rat, which all attack a hydrogen at the carbon atom proximal to the polar head. Behrouzian et al., Curr. Opin. Chem. Biol. 6 (2002) 577-582.

The plant sphingolipid Δ8-desaturases tested in a yeast sur2Δ-mutant strain cultured with or without 4-hydroxysphinganine required a C4-hydroxylated substrate, suggesting that Δ8-desaturation followed C4-hydroxylation to yield (8E/Z)-4-hydroxysphing-8-enines. However, nonhydroxylated (Z)- and (E)-sphing-8-enines are present in plant glucosylceramides which may point to a second sphingolipid D8-desaturase activity in plants required for the synthesis of (4E,8E/Z)-sphing-4,8-dienines. In fact, a second sphingolipid D8-desaturase sequence is present in B. officinalis and in A. thaliana (Accession Number NM_{—}130183). Expression of this second A. thaliana cDNA in a S. cerevisiae sur2Δ-mutant strain revealed a similar C4-hydroxy-preference of the enzyme, although traces of sphing-8-enine were also formed. These data are consistent with the high proportions of Δ8-unsaturated trihydroxybases but minor proportions of Δ8-unsaturated dihydroxybases present in A. thaliana sphingolipids. Recently, expression of a sphingolipid Δ8-desaturase from Aquilegia vulgaris in S. cerevisiae and in a sur2Δ-mutant showed that this enzyme is able to use both 4-hydrosphinganine and sphinganine as substrates, respectively. Michaelson et al., Biochem. Soc. Trans. 30 (2001) 1073-1075.

The LCB composition of this member of the Ranunculaceae has not been analysed yet. Therefore, it may be speculated that plant species containing higher proportions of (E/Z)-sphing-8-enine and (4E,8E/Z)-sphing-4,8-dienine express Δ8-desaturase isoenzymes differing in their selectivity for 4-hydroxysphinganine and sphinganine. Interestingly, expression of a moss cDNA in S. cerevisiae resulted in the first identification of a cis-specific sphingolipid Δ8-desaturase using 4-hydroxysphinganine as substrate. Furthermore, the activity of this Δ8-(Z)-desaturase is significantly increased in the presence of glucosylceramide. However, in plants, the sequence of hydroxylation and desaturation to form 4-hydroxysphing-8-enines and sphinga-4,8-dienes remains to be elucidated.

The sphingolipid D8-desaturase sequences identified in plants and fungi all show the histidine box motifs characteristic for membrane-bound desaturases and their desaturase domain is N-terminally fused to cytochrome b5. The identification of this N-terminal domain from sunflower has been confirmed by expression of the recombinant protein domain in exhibiting redox absorbance spectra characteristic for plant microsomal cytochrome b5.

3.2.4. Acyl Amide Hydroxylation

Sphingolipids from plants usually contain α-hydroxylated fatty acids. Evidence for a direct α-hydroxylation of fatty acyl residues when bound as elements of intact sphingolipids or free ceramide came from radiolabeling studies of Tetrahymena pyriformis. Kaya et al., J. Biol. Chem. 259 (1984) 3548-3553. A non-essential acyl amide α-hydroxylase gene, FAH1 or SCS7, respectively, has been identified in S. cerevisiae by gene disruption/deletion leading to a significant reduction in 2-hydroxylated cerotic acid (26h:0).

Database searches revealed orthologous sequences from S. pombe, Caenorhabditis elegans and A. thaliana. Heterologous expression of one of the two A. thaliana homologs found in the genome (i.e., for example, Accession Number AY050326), led to a significant increase in 26h:0 in a fah1D yeast mutant strain. Whether the second homolog (i.e., for example, Accession Number AY058151) codes for an isoenzyme of the acyl amide 2-hydroxylase or for a 3-hydroxylase, responsible for the formation of 2,3-hydroxylated acyl amides as mentioned above, remains to be determined. Interestingly, the yeast and C. elegans protein sequences each showed a N-terminal cytochrome b5-fusion which is lacking in the A. thaliana and S. pombe orthologs.

3.2.5. Phylogenetic Relationships

The four different groups of enzymes (i.e., for example, acyl amide ahydroxylase, LCB C4-hydroxylase, LCB Δ4-(E)-desaturase, or LCB Δ8-(E/Z)-desaturase) modifying the hydrophobic ceramide core may belong to a large superfamily of membrane bound proteins including, but not limited to, fatty acid desaturases involved in the biosynthesis of polyunsaturated fatty acids. These oxygen-dependent enzymes are characterized by three conserved histidine motifs which may be involved in binding a di-iron complex.

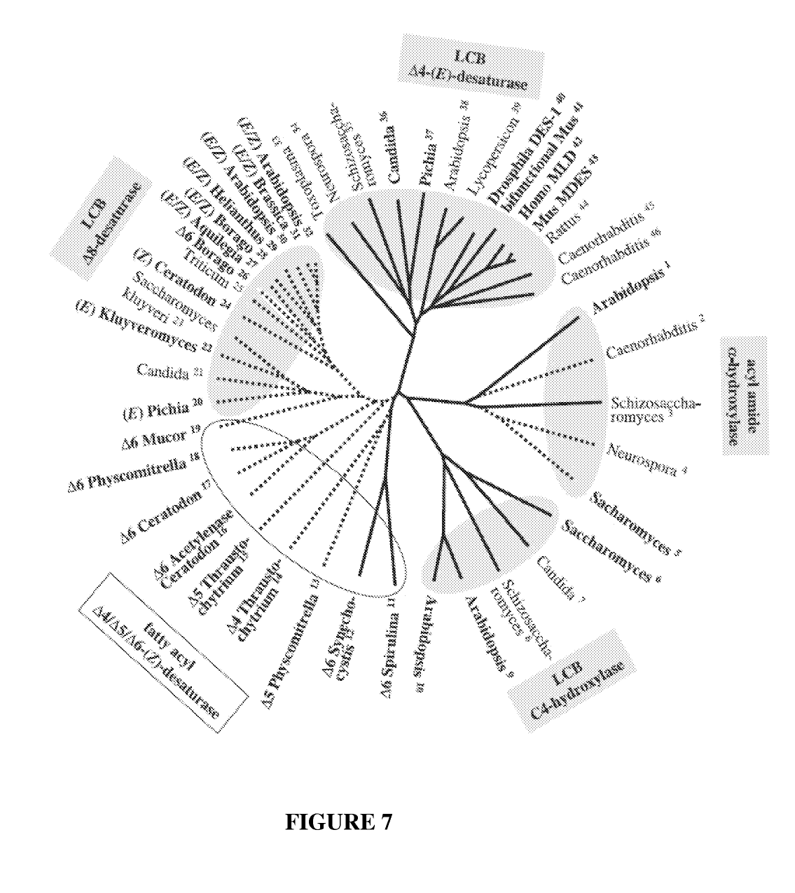

A phylogram derived from amino acid alignments of these sphingolipid desaturases and hydroxylases shows four distinct branches originating in the middle of the phylogram which indicates a very early separation of these paralogous groups. See, FIG. 7. It is assumed that enzymes with identical or similar regioselectivity are also similar in their amino acid sequence. Sperling et al., Eur. J. Biochem. 267 (2000) 3801-3811. Sphingolipid Δ8-desaturases are more similar to fatty acyl Δ5- and Δ6-desaturases all of which are cytochrome b5 fusion proteins and have evolved independently of sphingolipid Δ4-desaturase activity. Napier et al., Trends Plant Sci. 4 (1999) 2-4. Interestingly, the fatty acyl lipid Δ4-desaturase, which recently has been cloned from Thraustochytrium sp. and which is also a cytochrome b5-fusion protein, is also more similar to the fatty acyl Δ5/Δ6-desaturases than to the non-fused sphingolipid Δ4-desaturase and C4-hydroxylase. Qiu et al., J. Biol. Chem. 276 (2001) 31561-31566. Thus, Δ4/C4-regioselectivity must have evolved independently three times pointing to a convergent evolution of fatty acyl Δ4-desaturase, sphingolipid Δ4-desaturase and sphingolipid C4-hydroxylase. The invariant fusion between sphingolipid Δ8-desaturases and cytochrome b5 which represents the immediate electron donor for many microsomal desaturases, may have a functional advantage.

3.3. Formation of Complex Sphingolipids

One possible modification of the primary hydroxyl group of ceramides occurs by glycosylation yielding cerebrosides. cDNAs coding for UDP-glucose:ceramide β-D-glucosyltransferase (GCS) have not been found in either S. cerevisiae or S. pombe which is consistent with the lack of cerebrosides in these yeasts. Ichikawa et al., Proc. Natl. Acad. Sci. U.S. A. 93 (1996) 4638-4643; Takakuwa et al., FEMS Yeast Res. 2 (2002) 533-538; and Leipelt et al., Biochem. Soc. Trans. 28 (2000) 751-752. Recently, a plant GCS has been identified from Gossypium arboreum (cotton) by its functional expression in P. pastoris showing similarity to a putative GCS sequence (Accession Number AF424585) in A. thaliana. There is only little information on the substrate specificity of plant GCS. Studies on the mammalian enzyme showed that it requires UDP-glucose and N-acyl-D-erythrosphinganine and that it does not accept the L-erythro enantiomer or the L-threo diastereomer. Venkataraman et al., Biochim. Biophys. Acta 1530 (2001) 219-226. Unexpectedly, an in vitro assay using microsomal membranes from bean hypocotyls and radiolabeled sterol glucoside as substrate demonstrated UDP-glucose-independent GCS activity. Lynch et al., Arch. Biochem. Biophys. 340 (1997) 311-316. The assumption that sterol glucoside may function as a glucosyl donor is in line with recent data that sterol glucosides act as primers for cellulose synthesis in plants. Read et al., Science 295 (2002) 59-60. In contrast, a sterol glucosyltransferase/GCS double null-mutant of P. pastoris expressing the cotton GCS resulted in sterol glucoside-independent glucosylceramide synthesis, suggesting that UDP-glucose is the actual sugar donor. The presence of an N-terminal transmembrane domain and the sterol glucoside-independency of the plant enzyme supports a location in the Golgi apparatus as suggested for the mammalian enzyme rather than an exoplasmic orientation in the plasma membrane. Cantatore et al., Biochem. Soc. Trans. 28 (2000) 748-750.

Expression of the cotton GCS in a P. pastoris GCS-null mutant resulted in characteristic glucosylceramide species with non- and α-hydroxylated C₁₆- to C₂₄-acyl amides, suggesting that in fungi, initial Δ4-(E)-desaturation is followed by Δ8-(E)-desaturation and final C9-methylation of the LCB. The fact that (4E,8E)-9-methyl-sphinga-4,8-dienine is the major LCB in free ceramides of several fungi supports the assumption that ceramide glycosylation succeeds LCB modifications. Yaoita et al., Chem. Pharm. Bull. (Tokyo) 50 (2002) 681-684. However, it is not clear whether in plants, cerebroside formation occurs before or after the modifications of the hydrophobic ceramide core.

The formation of other complex plant sphingolipids such as mannosylceramide and GIPC is still unclear, because no gene coding for a mannosylceramide synthase or for an inositol phosphorylceramide synthase has been identified in plants yet. In S. cerevisiae, the first step in GIPC formation is catalyzed by the posphatidylinositol:ceramide phosphoinositol transferase (IPC synthase) which transfers the inositolphosphate moiety from phosphatidylinositol to the C-1 hydroxy group of ceramide. Becker et al., J. Bacteriol. 142 (1980) 747-754. The membrane-bound enzyme has been shown to be located in the Golgi apparatus of S. cerevisiae. Levine et al., Mol. Biol. Cell 11 (2000) 2267-2281. IPC synthase activity is inhibited by antifungal agents such as aureobasidin A, khafrefungin and rustmycin. In S. cerevisiae, the IPC synthase or a subunit of the enzyme is encoded by the AUR1 gene. Hashida-Okado et al., Mol. Gen. Genet. 251 (1996) 236-244; Nagiec et al., J. Biol. Chem. 272 (1997) 9809-9817. Surprisingly, a similar gene cannot be found in the A. thaliana genome or in other plant genomes such as rice, wheat, barley and oat. Due to the lack of any in vitro and in vivo studies or data concerning the effectiveness of the antifungal IPC synthase inhibitors in plant systems, there is no evidence for the existence of an IPC synthase in plants. Therefore, in plants, either a completely different (insensitive) protein or a different reaction mechanism may be involved in this step.

4. Functions of Sphingolipids in Plants

There is still little information on the functions of sphingolipids in plants. However, functions including, but not limited to, cell signaling, membrane stability, stress response, pathogenesis and apoptosis have been suggested.

4.1. Cell Signaling

Sphingolipids are thought to be cellular mediators not only in animals and fungi, but also in plants. For example, a knock-out of an A. thaliana gene coding for a protein accelerating in vitro (E)-sphing-4-enine transfer between membranes caused activation of cell death and defense-related genes. Brodersen et al., Genes Dev. 16 (2002) 490-502. Evidence for the presence of (E)-sphing-4-enine-1-phosphate in plants has been achieved showing its involvement in drought-induced signal transduction in guard cells linking the perception of abscisic acid to a reduction in turgor. Ng et al., Nature 410 (2001) 596-599. Furthermore, sphing-4-enine-1-phosphate promoting Ca²⁺-mediated guard cell closure required the presence of the Δ4-double bond. Therefore, the low abundance of sphing-4-enine in plant sphingolipids does not indicate its biological insignificance, but may point either to a selective incorporation into rare sphingolipids or to an exclusive occurrence in specific tissues or cell types, respectively. Sphingolipids may further play a role in the establishment and maintenance of cell polarity via control of the actin cytoskeleton and that accumulation of ceramide and is likely responsible for arresting the cell cycle in G1.

4.2. Membrane Stability

Sphingolipids may also be important for plant membrane organization. Mammalian and fungal sphingolipids have a tendency to associate with cholesterol or ergosterol, respectively, and form clusters of raft-like domains, which are important for lateral sorting of proteins, cellular trafficking and signal transduction. R. T. Dobrowsky, Cell. Signal. 12 (2000) 81-90; van Meer et al., J. Biol. Chem. (2002) 25855-25858; and Simons et al., Nat. Rev. Mol. Cell Biol. 1 (2000) 1-39. Plasma membrane microdomains exist in plant cells as well, although they contain sterols other than cholesterol. Peskan et al., Eur. J. Biochem. 267 (2000) 6989-6995. The plant-specific sterols sitosterol and stigmasterol, differing in aliphatic side chain structure from cholesterol, by an additional ethyl group at C-24 and the latter by an additional Δ22-(E)-double bond, also promote domain formation, which is modulated by sterol side chain structure. Xu et al., J. Biol. Chem. 276 (2001) 33540-33546. Moreover, small amounts of free ceramide significantly stabilize domain formation, suggesting that this signaling molecule is likely to concentrate within sphingolipid/sterol rafts. The presence of GPI-anchored proteins in plants have lead to the identification of a COB(RA)-like multigene family in A. thaliana encoding putative GPI-anchored proteins, which may be involved in orientated cell expansion at the plasma membrane-cell wall interface of vascular plants.

4.3. Abiotic Stress Response

Sphingolipids have been implicated in conferring stability to plant membranes, contributing to acclimation to drought stress and to cold hardiness in chilling resistant plants Lynch et al., Plant Physiol. 83 (1987) 761-767; Bohn, PhD thesis, Faculty of Biology, University of Hamburg, Hamburg, Germany, (1999). It has been observed that the proportion of glucosylceramides in plasma membranes of freezing-tolerant plants is lower than in freezing-sensitive plants and that the glucosylceramide content is reduced following cold acclimation. The molecular species composition of cerebrosides differed among chilling-sensitive and -tolerant plants and changed during cold acclimation. Among many plant species analysed, hydroxy nervonic acid (24h:1) was only found in the leaf cerebrosides of chilling-resistant plants, suggesting that cerebroside species with monounsaturated very long-chain hydroxy acyl amides exhibit much lower phase transitions than those having a saturated hydroxy acyl amide residue. Furthermore, most of the chilling-resistant plant species analysed had more 8-(Z)-unsaturated than 8-(E)-trihydroxybases, suggesting that high levels of (8E/Z)-4-hydroxysphing-8-enine are correlated with freezing tolerance.

4.4. Phytopathogenesis

A new field of sphingolipid functions comprises plant-pathogen interactions. For example, certain cerebrosides isolated from plants stimulate fruiting body formation of Schizophyllum commune, a fungus involved in wood degradation. The active glucosylceramides from wheat grain consisted of α-hydroxylated C₁₆- or C₁₈-acyl amides, and of (4E,8Z)-sphinga-4,8-dienine or (Z)-sphinga-8-enine. Kawai et al., J. Biol. Chem. 261 (1986) 779-784. Hydrogenation of the (Z)-sphinga-8-enine containing glucosylceramide showed that the stimulatory effect of these cerebrosides was dependent on the presence of a 48-double bond. More recent studies showed that fungal cerebrosides function as elicitors causing hypersensitive cell death, phytoalexin accumulation and increased resistance to subsequent infections by compatible pathogens in plants. Elicitor-active glucosylceramides were isolated from the rice pathogenic fungus Magnaporthe grisea having an amide-linked (3E)-2-hydroxyhexadec-3-enoyl or (3E)-2-hydroxyoctadec-3-enoyl group bound to (4E,8E)-9-methyl-sphinga-4,8-dienine. Interestingly, hydrogenation of the Δ8-(E)-double bond in the LCB or of the Δ3-(E)-double bond in the acyl amide moiety of the cerebroside did not alter elicitor activity, whereas the Δ4-(E)-double bond of the LCB and the methyl group at C-9 were essential for elicitor activity. The glucose headgroup was not crucial, because free ceramide also showed elicitor activity though with reduced effectiveness In field experiments with application of as little as 3×45 g/ha, these glucosylceramide elicitors, which occur in many different phytopathogens, protected rice plants against M. grisea and other diseases as well, indicating that cerebrosides function as general elicitors in a wide range of rice-pathogen interactions. The importance of the Δ8-double bond in fungal fruiting body induction or of the Δ4-double bond and 9-methyl group in the hypersensitive response to phytopathogens indicates that diverse structural LCB modifications are contributing to different cellular responses in plant-pathogen interactions. The observation of an increase in the expression of serine palmitoyltransferase during the hypersensitive response of a late-blight-resistant potato to Phytophthora infestans also points to the involvement of sphingolipids in pathogenesis. Koga et al., Biol. Chem. 273 (1998) 31985-31991; Uemura et al., Plant Cell Physiol. 41 (2000) 676-683; Uemura et al., Plant Cell Physiol. 43 (2002) 778-784; Birch et al., Mol. Plant-Microb. Interact. 12 (1999) 356-361.

4.5. Programmed Cell Death

Sphingolipids may be involved in plant apoptosis and ceramide signalling as sphinganine-analogous mycotoxins (SAMs) such as fumonisins produced by Fusarium moniliforme and related fungi, and host-selective toxins secreted from Alternaria alternate f sp. Lycopersici (AAL), have been shown to be cytotoxic and cancerogenic in animals. SAMs have been shown to induce necrosis, DNA fragmentation and accumulation of free LCB in different plant tissues. Siler et al., Physiol. Mol. Plant Pathol. 23 (1983) 265-274; Abbas et al., Plant Physiol. 106 (1994) 1085-1093; Wang et al., Plant Cell 8 (1996) 375-391; Moore et al., Physiol. Mol. Plant Pathol. 54 (1999) 73-85. These results could be attributed to a competitive inhibition of the sphinganine N-acyltransferase (ceramide synthase) activity. Merrill Jr., et al., In: D. E. Vance, J. E. Vance (Eds.), Biochemistry of Lipids, Lipoproteins and Membranes, vol. 31, Elsevier, Amsterdam, 1996, pp. 309-339; D. G. Gilchrist, Cell Death Differ. 4 (1997) 689-698; P. V. Minorsky, Plant Physiol. 129 (2002) 929-930.

In tomato, co-dominant insensivity to SAMs is believed mediated by the ASC1 gene (Alternaria stem canker), which is homologous to the yeast longevity assurance gene (LAG1) and facilitates the ER-to-Golgi transport of GPI-anchored proteins. Overexpression of Asc1 in SAM-sensitive plants resulted in resistance to infection by A. alternata f sp. lycopersici, indicating that susceptibility of tomatoes for SAMs may involve sphingolipids and ER-to-Golgi transport of GPI-anchored proteins. Labeling experiments of Asc/Asc and asc/asc tomato leaf discs with tritiated serine indicated that the presence of Asc1 is able to relieve an AAL toxin-induced block of sphingolipid synthesis that otherwise would lead to programmed cell death. Spassieva et al., Plant J. 32 (2002) 561-572.

B. Morphine Analgesia and Sphingolipids

Morphine and many other opioid agonists have analgesic effects that are believed to be mediated by their activation of inhibitory opioid receptors on nociceptive (pain-mediating) neurons. Accordingly, these opioids are administered to relieve severe pain. Morphine and many other opioid agonists, however, also have been shown to activate excitatory opioid receptors on nociceptive neurons, thereby attenuating the analgesic potency of the opioid agonists, and resulting in the development of anti-analgesia, hyperexcitability, hyperalgesia, physical dependence, psychological dependence, tolerance, and other adverse (excitatory) effects Crain et al.,” Opioids can evoke direct receptor-mediated excitatory effects on sensory neurons” Trends in Pharmacol. Sci., 11:77-81 (1990). Consequently, a long-standing need has existed to develop a method which will both enhance the analgesic (inhibitory) effects of these bimodally-acting opioid agonists and block or prevent adverse (excitatory) effects associated with their administration.

It has been reported that the analgesic potency of bimodally-acting opioid agonists can be enhanced, and the tolerance/dependence liability reduced, by co-administering bimodally-acting opioid agonists with ultralow doses of selective excitatory opioid receptor antagonists (e.g., U.S. Pat. Nos. 5,472,943; 5,512,578; 5,580,876; and 5,767,125, all herein incorporated by reference). Excitatory opioid receptor antagonists are compounds that bind to and inactivate excitatory opioid receptors, but not inhibitory opioid receptors, on neurons in nociceptive (pain) pathways. Selective excitatory opioid receptor antagonists attenuate excitatory, but not inhibitory, opioid receptor functions in nociceptive pathways of the peripheral and central nervous systems. As a result, symptoms associated with activation of excitatory opioid receptors (e.g., anti-analgesia, hyperalgesia, hyperexcitability, physical dependence, and tolerance effects) are blocked, while the analgesic effects of the bimodally-acting opioid agonists, which are mediated by the inhibitory opioid receptors, are unmasked and thereby enhanced. Crain et al., “Ultra-low concentrations of naloxone selectively antagonize excitatory effects of morphine on sensory neurons, thereby increasing its antinociceptive potency and attenuating tolerance/dependence during chronic cotreatment” Proc. Natl. Acad. Sci. USA, 92:540-544 (1995); Crain et al., “GM1 ganglioside-induced modulation of opioid receptor-mediated functions” Ann. N.Y. Acad. Sci., 845:106-125 (1998); Crain et al., “Modulation of opioid analgesia, tolerance and dependence by Gₛ-coupled, GM1 ganglioside-regulated opioid receptor functions” Trends in Pharmacol. Sci., 19:358-65 (1998). These preclinical studies have suggested that ultralow doses of selective excitatory opioid receptor antagonists can be administered alone to chronic pain patients to enhance the analgesic potency and reduce the tolerance/dependence liability of endogenous opioid peptides, such as enkephalins, dynorphins, and endorphins, which are elevated in chronic pain patients.

It has also been reported that ultralow doses of naltrexone, alone or in combination with low-dose methadone (e.g., U.S. Pat. No. 5,512,578, herein incorporated by reference), and ultralow doses of other excitatory opioid receptor antagonists alone (e.g., U.S. Pat. Nos. 5,580,876 and 5,767,125, both herein incorporated by reference), can provide effective, long-term maintenance treatment for opioid addiction after acute detoxification, and can prevent relapse to drug abuse.

GM1 is a monosialoganglioside that is abundantly distributed on the external surface of neuronal cell membranes. Fishman, P. H., “Gangliosides and cell surface receptors and transducers of biological signals” In: New Trends in Ganglioside Research: Neurochemical and Neuroregenerative Aspects, R. W. Ledeen, E. L. Hogan, G. Tettamenti, A. J. Yates, and R. K. Yu (eds.) (Padova: Liviana, 1988) pp. 183-201. GM1-ganglioside plays a role in regulating excitatory opioid receptors in nociceptive neurons, probably by binding to an allosteric regulatory site on opioid receptors. Shen et al., “Cholera toxin-B subunit blocks opioid excitatory effects on sensory neuron action potentials indicating that GM1 ganglioside may regulate Gₛ-linked opioid receptor functions” Brain Res. 531:1-7 (1990); and Shen et al., “Brief treatment of sensory ganglion neurons with GM1 ganglioside enhances the efficacy of opioid excitatory effects on the action potential” Brain Res., 550:130-138 (1991). Other data have suggested that the analgesic potency of opioid agonists is enhanced, and the tolerance/dependence liability of endogenous opioid peptides is reduced, when opioids are co-administered with high doses of exogenous GM1-ganglioside. Mao et al., “Thermal hyperalgesia in association with the development of morphine tolerance in rats: roles of excitatory amino acid receptors and protein kinase C” J. Neurosci. 14:2301-2312 (1994); Mayer et al., “The development of morphine tolerance and dependence is associated with translocation of protein kinase C” Pain 61:365-374 (1995); U.S. Pat. Nos. 5,321,012; 5,502,058; 5,556,838; and 5,654,281 (all herein incorporated by reference).

In contrast, it has demonstrated that intraperitoneal (i.p.) injection of low doses of exogenous GM1-ganglioside (0.1 mg/kg) in mice rapidly attenuates morphine's analgesic effects. Crain et al., “Enhanced analgesic potency and reduced tolerance of morphine in 129/SvEv mice: evidence for a deficiency in GM1 ganglioside-regulated excitatory opioid receptor functions” Brain Res., 856:227-235 (2000). As a result, it was suggested that the analgesic potency of morphine may be increased upon co-administration with cholera toxin B (CTX-B) or oseltamivir. Both these agents inhibit specific functions of endogenous GM1-ganglioside on opioid receptors in nociceptive neurons. Crain et al. “Methods for increasing analgesic potency and attenuating adverse excitatory effects of bimodally-acting opioid agonists by inhibiting GM1-ganglioside” United States Patent Application 2002/0137761. Cholera toxin consists of a non-covalently-assembled pentamer of a non-toxic B subunit (CTX-B) that is responsible for cell attachment, and a toxicogenic A subunit (CTX-A). Mekalanos et al., “Cholera toxin gene: nucleotide sequence, deletion analysis and vaccine development” Nature, 306:551-557 (1983). CTX-B binds selectively to GM1-ganglioside on the surface of cells, and facilitates penetration of CTX-A into the cell membrane. Gill, D. M., “Mechanism of action of cholera toxin” Adv. Cycl. Nucl. Res., 8:85-118 (1977).

Oseltamivir is an ethyl ester pro-drug that requires ester hydrolysis for conversion to the active form, oseltamivir carboxylate. The proposed mechanism of action of oseltamivir is via inhibition of influenza-virus neuraminidase, with the possibility of alteration of virus particle aggregation and release. Oseltamivir and its analogues and derivatives are available commercially. Oseltamivir is prepared in tablet form, for oral administration, under the trademark Tamiflu®, and may be obtained from Roche Laboratories (Nutley, N.J.). Tamiflu® is available as a capsule containing 75 mg of oseltamivir for oral use, in the form of oseltamivir phosphate. Tamiflu® may be administered to a subject in a dose ranging from 0.1-1 mg/kg, once or twice a day.

Oseltamivir at doses that result in neuraminidase inhibition of influenza virus also may be effective in decreasing GM1-ganglioside levels in nociceptive neurons. Such a decrease in levels of GM1-ganglioside would result in attenuation of the efficacy of GM1-regulated, Gₛ-coupled, excitatory opioid receptor-mediated hyperalgesic functions, thereby unmasking Gᵢ/Gₒ-coupled inhibitory opioid receptor-mediated analgesia and reducing development of tolerance and physical dependence. Gubareva et al., “Influenza virus neuraminidase inhibitors” Lancet 355:827-835 (2000).

Previous studies have shown that pretreatment of dorsal root ganglion (DRG) neurons with CTX-B selectively blocks opioid-induced prolongation of the action potential duration (APD), but not opioid-induced shortening of the APD, suggesting that GM1-ganglioside may regulate Gₛ-linked excitatory opioid receptor functions in DRG neurons. Shen et al., “Cholera toxin-B subunit blocks opioid excitatory effects on sensory neuron action potentials indicating that GM1 ganglioside may regulate Gₛ-linked opioid receptor functions” Brain Res., 531:1-7 (1990). Additional studies on DRG neurons in culture have shown that chronic opioid treatment, together with administration of nanomolar concentrations of CTX-B, prevents development of tolerance to the inhibitory, APD-shortening effects of micromolar concentrations of the opioid, by preventing the development of opioid excitatory supersensitivity (i.e., for example, a cellular manifestation related to opioid tolerance and dependence in vivo). Shen et al., “Chronic selective activation of excitatory opioid receptor functions in sensory neurons results in opioid “dependence” without tolerance” Brain Res., 597:74-83 (1992). CTX-B blockade appears to involve interference with GM1-ganglioside regulation of opioid excitatory receptor functions. In particular, it was reported that CTX-B binds with selective high affinity (K_{D}=10⁻¹⁰ M) to GM1-ganglioside. Wu et al., “The role of GM1 ganglioside in regulating excitatory opioid effects” Ann. N.Y. Acad. Sci., 845:126-138 (1998). Treatment of DRG neurons with anti-GM1 antibodies has been shown selectively to block opioid-induced APD prolongation, as occurs with CTX-B. Shen et al., “Cholera toxin-B subunit blocks opioid excitatory effects on sensory neuron action potentials indicating that GM1 ganglioside may regulate Gₛ-linked opioid receptor functions” Brain Res., 531:1-7 (1990).

Gangliosides are a class of galactose-containing complex glycolipids (i.e., for example, sphingolipids). Gangliosides are found in highest concentration in the nervous system, particularly in gray matter, where they constitute 6% of the lipids. In gangliosides, an oligosaccharide chain containing at least one acidic sugar is attached to ceramide. The acidic sugar is N-acetylneuraminate or N-glycolylneuraminate, both of which are sialic acids. GM1 is a monosialoganglioside that is abundantly distributed on the external surface of neuronal cell membranes. It is formed by the addition of N-acetylgalactosamine and a galactose group.

Neuraminidase also regulates cellular levels of GM1-ganglioside. For example, administration of exogenous neuraminidase has been shown markedly to increase the concentrations of GM1-ganglioside in cell membranes of DRG cells and other neurons by enzymatic removal of neuraminic (sialic) acid from polysialylated ligands of the gangliotetraose series. Wu et al., “Stimulation of neurite outgrowth in neuroblastoma cells by neuraminidase: putative role of GM1 ganglioside in differentiation” J. Neurochem. 56:95-104 (1991); and Wu et al., “GM1 ganglioside modulates prostaglandin E1 stimulated adenylyl cyclase in neuro-2A cells” Glycoconjugate J., 13:235-239 (1996); , 64, 65, 69). This specific effect of neuraminidase on the enzymatic conversion of polysialylated gangliosides to GM1-ganglioside in neurons is quite distinct from the roles of neuraminidase in promoting release of influenza virus from infected cells and in facilitating virus spread within the respiratory tract.

In one embodiment, an agent that inhibits GM1-ganglioside in nociceptive neurons may be a neuraminidase inhibitor, including, but not limited to, MgSO₄, Na₂SO₄, oseltamivir, or zanamivir. For example, Na₂SO₄ may be administered to a subject in need of treatment for pain in an amount on the order of 10 mg/kg per day.

### VII. Pharmaceutical Formulations

The present invention further provides pharmaceutical compositions (e.g., comprising the Kratom-dervied compounds described above). The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary (e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration.

Pharmaceutical compositions and formulations for topical administration may include, but are not limited to, transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include, but are not limited to, powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include, but are not limited to, sterile aqueous solutions that may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances that increase the viscosity of the suspension including, for example, sodium carboxymethylcellulose, sorbitol and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies and liposomes. While basically similar in nature these formulations vary in the components and the consistency of the final product.

The compositions of the present invention may additionally contain other adjunct components conventionally found in pharmaceutical compositions. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like.

Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. The administering physician can easily determine optimum dosages, dosing methodologies and repetition rates. Optimum dosages may vary depending on the relative potency of individual compounds, and can generally be estimated based on EC₅₀'s found to be effective in in vitro and in vivo animal models or based on the examples described herein. In general, dosage is from 0.01 mg to 100 g per kg of body weight, and may be given once or more daily, weekly, monthly or yearly. The treating physician can estimate repetition rates for dosing based on measured residence times and concentrations of the drug in bodily fluids or tissues. Following successful treatment, it may be desirable to have the subject undergo maintenance therapy to prevent the recurrence of the disease state, wherein the compound is administered in maintenance doses, ranging from 0.01 μg to 100 g per kg of body weight, once or more daily, to once every 20 years.

### EXPERIMENTAL

### Example I

### Withdrawal from Hydromorphone Addiction

This example shows that long-term Kratom use promotes extinction of opioid cravings in a middle aged male, who, after transitioning without withdrawal from injection hydromorphone use to Kratom administration. The patient was adminstered Kratom for three years and subsequently terminated Kratom use without a taper (i.e., for example, a successive reduction in daily dose to effectively zero). The patient did not exhibit any symptoms of opioid withdrawal except for rhinorrhea.

A 43-year-old male was admitted for evaluation of a generalized tonic-clonic seizure. The patient's medical history included chronic pain from thoracic outlet syndrome that was being treated with hydromorphone. As hydromorphone-induced tolerance escalated, the patient began injecting 10 mg hydromorphone per day (S.C.) from crushed pills. When hydromorphone was unavailable, the patient purchased Kratom (i.e., for example, from internet vendors) and self-administered the compound (˜$15K per year).

Following one such abrupt hydromorphone cessation (3.5 years before seeking professional substance abuse treatment) the patient averted opioid withdrawal by ingesting a tea made from Kratom four times a day. The patient attributed substantial pain relief to Kratom as well as improved alertness and did not experience drowsiness often accompanied by opioid use.

The patient then co-administered 100 mg modafinil with Kratom in an attempt to further improve alterness. Twenty minutes following this co-admininstration, he experienced a generalized tonic-clonic seizure lasting 5 minutes. Vital signs at presentation were: pulse 123 beats per minute, blood pressure 130/74 mm/Hg, respiratory rate 16; he was afebrile. After a brief post-ictal period, his physical examination was normal except for meiosis. He had no previous history of seizures or head trauma, and he denied alcohol or recent illicit drug abuse. Laboratory studies were unremarkable; qualitative urine drugs of abuse and comprehensive toxicology screening identified only modafinil. Computerized tomography and magnetic resonance imaging of the brain were normal. We identified no adulterants or contaminants. Upon discharge, the patient abruptly ceased use of Kratom and sought the care of an addiction specialist.

This withdrawal from Kratom was considerably less intense, but more protracted, than that previously experienced from prescription opioids. Physician-observed features of Kratom included rhinorrhea, insomnia, poor concentration, constricted affect and myalgias persisting for 10 days from his last dose of kratom. To prevent relapse, an addiction specialist prescribed buprenorphine/naloxone, reaching a maintenance dose of 16 mg per day. Rhinorrhea ceased on the first day of suboxone therapy.

The patient currently reports adequate pain control, and follow-up urine screens for drugs of abuse have remained negative. We confirmed the identity of the plant matter ingested by the patient as Kratom by comparison against a known standard (Pure Land Ethnobotanicals, Madison, Wis., USA) utilizing existing extraction and high-performance liquid chromatography protocols.

### Example II

### Mitragynine Pharmacokinetics

After a 12 h fast, rats (n=8 per sampling time) received by gavage a single oral dose of 20 mg/kg mitragynine dissolved in 1% acetic acid pH 4.7 (adjusted with 1M NaOH solution).³⁹ Heparinzed blood samples were collected at times zero, 0.5, 1, 2, 3, 4, 5, 6, 8, 10, 15, 24 and 48 hr after administration of the drug; detection was performed using liquid chromatography-tandem mass spectroscopy with electrospray interface.

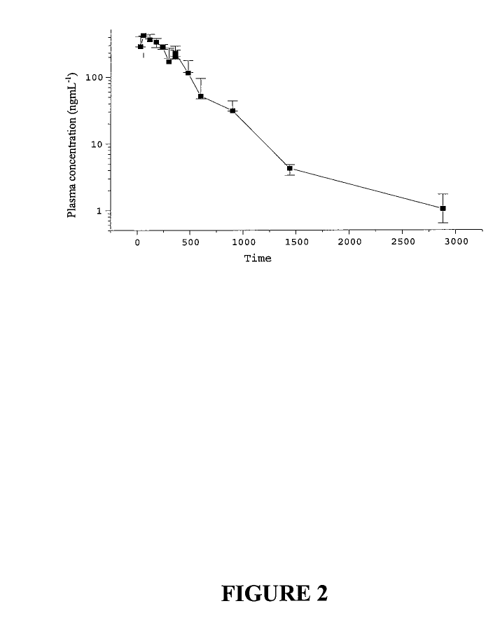

Pharmacokinetic parameters were calculated based on the plasma concentration versus time curves using WinNonlin version 4.0 program using first-order kinetics, a monocompartment model and no lag time. The experimental data were analyzed statistically using the Graphpad Instat® software for the calculation of mean, median, and 95% confidence interval. The obtained pharmacokinetic data is presented graphically. See, FIG. 2.

### Example III

### Validation Of Mitragynine Detection and Stability

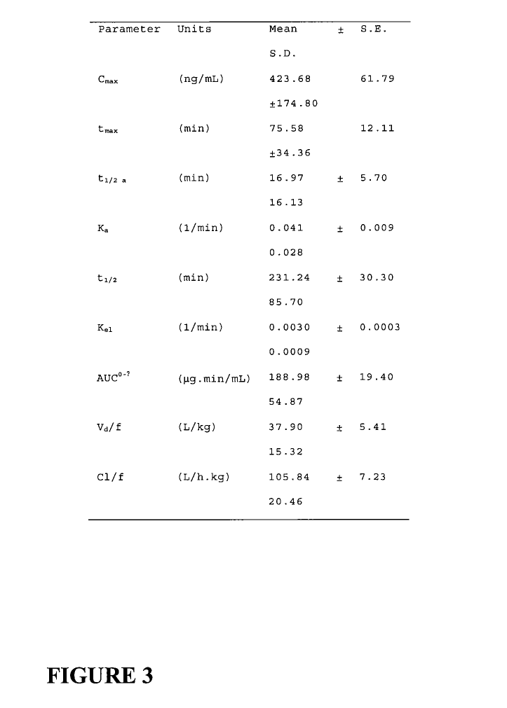

The method developed for detection of mitragynine has been validated by evaluating recovery, linearity, precision, accuracy, quantification limit, and stability. The coefficient of variation and relative error values at the three concentrations were obtained in the intra- and inter-day assays. See, FIG. 3. The results show that the analytical method is accurate (±15% different from the nominal concentration) and the precision values expressed as coefficient of variation are within the accepted limits of 15% or less for the concentration levels studied. Furthermore, tests of short-term (4 h at room temperature), freeze-thaw (3 cycles) and post-processing (16 h at 12° C.) stabilities demonstrated sample stability with no variation of more than 15% at any of the concentrations tested.

### Example IV

### Effects of Kratom Extracts on Morphine Withdrawal Symptoms

In this experiment mice were chronically administered either morphine using an increasing dose paradigm starting at 20 mg/kg and ending at 100 mg/kg (i.p.) or a saline vehicle control for six days. Subsequent to the morphine administration the mice were administered either: i) Kratom tea extract (1 g/kg; p.o.); ii) Kratom tea extract (2 g/kg; p.o.); iii) methadone (23 mg/kg; p.o.); iv) methadone (100 mg/kg; p.o.); or v) mitragynine (23 mg/kg; p.o.) for another six days. Morphine withdrawal symptoms were initiated by the injection of naloxone (10 mg/kg; i.p.). Kratom extract and mitragyine reduced naloxone-precipitated morphine withdrawal symptoms in a similar manner as methadone. The Kratom extract administrations demonstrated a dose-dependent effect.

TABLE 1
Effect of oral administration of lyophilized tea extract of Mitragyna speciosa and pure
mitragynine on precipitation of morphine withdrawal symptoms.

| Morphine | | | | | | |
|---|---|---|---|---|---|---|
| (20-100 mg/kg) | | | | | | |
| (i.p.) + | | | | | | |
| Compound + | | | | Wet Dog | Teeth | Last Day % |
| 10 mg/kg Naloxone | Locomotor | Jumping | Paw Tremor | Shakes | Chattering | Weight |
| (i.p.) | Activity | Frequency | Frequency | Frequency | Frequency | Control |
| Saline (i.p) | 3793 ± 150.0 | 2.700 ± 0.9551 | 0.00 ± 0.00 | 0.00 ± 0.00 | 0.00 ± 0.00 | 107.2 ± 1.393 |
| Morphine | 1870 ± 114.2*** | 21.6 ± 4.378*** | 156.2 ± 7.124*** | 9.433 ± 1.334*** | 76.10 ± 6.572*** | 89.64 ± 0.6012*** |
| (i.p.) | | | | | | |
| 1 g/kg | 2441.0 ± 222.4***^{#} | 0.00 ± 0.00^{###} | 9.667 ± 2.102^{###} | 10.78 ± 2.379*** | 0.8889 ± 0.6111^{###} | 96.61 ± 2.264***^{##} |
| Kratom Tea | | | | | | |
| Extract (p.o.) | | | | | | |
| 2 g/kg | 3045 ± 191.8*^{###} | 1.300 ± 0.5588^{###} | 3.200 ± 1.123^{###} | 7.500 ± 0.9339* | 0.00 ± 0.00^{###} | 92.92 ± 2.326*** |
| Kratom Tea | | | | | | |
| Extract (p.o.) | | | | | | |
| 23 mg/kg | 1866 ± 225.9*** | 0.00 ± 0.00^{###} | 110.7 ± 8.809***^{###} | 5.600 ± 0.9684* | 81.20 ± 8.704*** | 100.1 ± 0.8121^{###} |
| Methadone | | | | | | |
| (p.o.) | | | | | | |
| 100 mg/kg | 2512 ± 131.5***^{#} | 1.111 ± 0.7536^{###} | 8.444 ± 1.692^{###} | 0.6667 ± 0.3333^{###} | 0.00 ± 0.00^{###} | 100.1 ± 1.205^{###} |
| Methadone | | | | | | |
| (p.o.) | | | | | | |
| 23 mg/kg | 2485 ± 119.1***^{#} | 0.00 ± 0.00^{###} | 3.200 ± 0.7424^{###} | 0.6000 ± 0.2667^{###} | 0.00 ± 0.00^{###} | 101.3 ± 0.8619^{###} |
| Mitragynine | | | | | | |
| (p.o.) | | | | | | |

*p < 0.05,
**p < 0.001,
***p < 0.0001 verses Saline control (Dunnett's post hoc test)
^{#}p < 0.05,
^{##}p < 0.001,
^{###}p < 0.0001 verses Morphine control (Dunnett's post hoc test)

The control values for each compound administered in combination with naloxone is shown in Table 2.

TABLE 2
Effect of oral administration of lyophilized tea extract of Mitragyna speciosa and pure
mitragynine followed by naloxone administration in comparison to morphine and methadone

| | | | | | | Last |
|---|---|---|---|---|---|---|
| Compound + | | | Paw | Wet Dog | Teeth | Day % |
| 10 mg/kg Naloxone | Locomotor | Jumping | Tremor | Shakes | Chattering | Weight |
| (i.p.) | Activity | Frequency | Frequency | Frequency | Frequency | Control |
| Saline (i.p.) | 3616 ± 172.9 | 1.600 ± 0.8844 | 0.00 ± 0.00 | 0.00 ± 0.00 | 0.00 ± 0.00 | 103.6 ± 1.566 |
| Morphine | 1870 ± 114.2*** | 21.6 ± 4.378*** | 156.2 ± 7.124*** | 9.433 ± 1.334*** | 76.10 ± 6.572*** | 89.64 ± 0.6012*** |
| (i.p.) | | | | | | |
| Naloxone | 3291 ± 145.3^{###} | 1.800 ± 0.9978^{###} | 3.800 ± 0.9978^{###} | 0.6000 ± 0.3399^{###} | 0.00 ± 0.00^{###} | 99.94 ± 1.225^{###} |
| (i.p.) | | | | | | |
| 1 g/kg | 2792 ± 181.6^{###} | 0.0700 ± 0.4726^{###} | 0.00 ± 0.00^{###} | 0.00 ± 0.00^{###} | 0.00 ± 0.00^{###} | 100.9 ± 1.786^{###} |
| Kratom Tea | | | | | | |
| Extract (p.o.) | | | | | | |
| 2 g/kg | 2747 ± 172.9**^{##} | 0.00 ± 0.00^{###} | 1.800 ± 0.9978*** | 8.800 ± 2.641*** | 0.00 ± 0.00^{###} | 92.39 ± 1.710*** |
| Kratom Tea | | | | | | |
| Extract (p.o.) | | | | | | |
| 23 mg/kg | 2595 ± 288.4**^{#} | 0.00 ± 0.00^{###} | 6.400 ± 1.462^{###} | 2.000 ± 0.5164^{###} | 0.00 ± 0.00^{###} | 96.64 ± 1.046* |
| Methadone | | | | | | |
| (p.o.) | | | | | | |
| 100 mg/kg | 2.886 ± 228.4*^{###} | 0.00 ± 0.00^{###} | 5.000 ± 1.549^{###} | 1.900 ± 0.5859^{###} | 0.00 ± 0.00^{###} | 98.30 ± 0.6976^{###} |
| Methadone | | | | | | |
| (p.o.) | | | | | | |
| 23 mg/kg | 3010 ± 108.1^{###} | 0.00 ± 0.00^{###} | 1.800 ± 0.6464^{###} | 0.00 ± 0.00^{###} | 0.00 ± 0.00^{###} | 98.85 ± 0.8938^{###} |
| Mitragynine | | | | | | |
| (p.o.) | | | | | | |
| Vehicle | 3172 ± 189.7^{###} | 0.00 ± 0.00^{###} | 2.300 ± 0.6839^{###} | 0.9000 ± 0.3480^{###} | 0.00 ± 0.00^{###} | 99.66 ± 0.9439^{###} |
| (1:1:18) | | | | | | |
| (p.o.) | | | | | | |

*p < 0.05,
**p < 0.001,
***p < 0.0001 verses Saline control (Dunnett's post hoc test)
^{#}p < 0.05,
^{##}p < 0.001,
^{###}p < 0.0001 verses Morphine control (Dunnett's post hoc test)

I claim:

1. A method, comprising:
a) providing,
i) a subject experiencing at least one withdrawal symptom induced by at least one addictive compound;
ii) a Kratom extract capable of reducing or preventing said at least one withdrawal symptom; and
b) administering said Kratom extract to said subject under conditions such that said at least one withdrawal symptom is prevented or reduced.

2. The method of claim 1, wherein said addictive compound comprises an opiate compound.

3. The method of claim 1, wherein said addictive compound comprises a cannabinoid compound.

4. The method of claim 1, wherein said addictive compound comprises an ethanol compound.

5. The method of claim 1, wherein said addictive compound comprises a cocaine compound.

6. The method of claim 2, wherein said opiate compound comprises a prescribed opiate compound.

7. The method of claim 1, wherein said Kratom extract comprises mitragynine.

8. The method of claim 1, wherein said Kratom extract comprises a mitragynine derivative.

9. The method of claim 8, wherein said mitragynine derivative comprises mitragynine pseudoindoxyl or 7-hydroxymitragynine.

10. The method of claim 1, wherein said withdrawal symptom comprises a craving induced by said addictive compound binding to a receptor selected from the group consisting of mu-, delta-, or kappa-opiate receptor.

11. A method, comprising:
a) providing,
i) a subject repeatedly exposed to at least one addictive compound, wherein said subject is at risk of experiencing at least one withdrawal symptom;
ii) a composition comprising mitragynine, wherein said composition is capable of reducing or preventing said at least one withdrawal symptom; and
b) administering said composition to said subject under conditions such that said at least one withdrawal symptom is prevented or reduced.

12. The method of claim 11, wherein said administering further comprises a regimen of decreasing said composition comprising mitragynine over a predetermined length of time.

13. The method of claim 11, wherein said addictive compound comprises an opiate compound.

14. The method of claim 11, wherein said addictive compound comprises a cannabinoid compound.

15. The method of claim 11, wherein said addictive compound comprises an ethanol compound.

16. The method of claim 11, wherein said addictive compound comprises a cocaine compound.

17. The method of claim 13, wherein said opiate compound comprises a prescribed opiate compound.

18. The method of claim 11, wherein said composition comprises a mitragynine derivative.

19. The method of claim 18, wherein said mitragynine derivative comprises mitragynine pseudoindoxyl or 7-hydroxymitragynine.

20. The method of claim 11, wherein said withdrawal symptom comprises a craving induced by said addictive compound binding to a receptor selected from the group consisting of mu-, delta-, or kappa-opiate receptor.

21. A method, comprising:
a) providing;
i) a subject repeatedly exposed to at least one addictive opioid compound, wherein said subject is at risk of experiencing at least one opioid withdrawal symptom;
ii) a composition comprising mitragynine, wherein said composition is capable of reducing or preventing said at least one withdrawal symptom; and
administering said composition to said subject under conditions such that said at least one withdrawal symptom is prevented and/or reduced.

22. The method of claim 21, wherein said administering further comprises a regimen of decreasing said composition comprising mitragynine over a predetermined length of time.

23. The method of claim 21, wherein said opiate compound comprises a prescribed opiate compound.

24. The method of claim 21, wherein said composition further comprises a mitragynine derivative.

25. The method of claim 24, wherein said mitragynine derivative comprises mitragynine pseudoindoxyl or 7-hydroxymitragynine.

26. The method of claim 21, wherein said withdrawal symptom comprises a craving induced by said addictive compound binding to a receptor selected from the group consisting of mu-, delta-, or kappa-opiate receptor.
